(19) United States
(12) Patent Application Publication
(10) Pub. No.: US 2024/0387009 A1
(43) Pub. Date: Nov. 21, 2024

(54) PREDICTING PERFORMANCE OF CLINICAL TRIAL SITES USING FEDERATED MACHINE LEARNING

(71) Applicant: Janssen Research & Development, LLC, Raritan, NJ (US)

(72) Inventors: Kaitlin Ann Hood, Philadelphia, PA (US); Francisco Xavier Talamas, Livingston, NJ (US); Geoffrey Jerome Kip, Philadelphia, PA (US); Hans Roeland Geert Wim Verstraete, Shilde (BE)

(21) Appl. No.: 18/689,859

(22) PCT Filed: Sep. 9, 2022
(86) PCT No.: PCT/IB2022/058523

§ 371 Date: Mar. 6, 2024

Related U.S. Application Data
(60) Provisional application No. 63/242,756, filed on Sep. 10, 2021.

(51) Int. Cl. G16H 10/20

(52) U.S. Cl. CPC G16H 10/20

(57) ABSTRACT

Disclosed herein are methods for predicting performance of one or more clinical trial sites for a prospective clinical trial, including obtaining input values of a plurality of clinical operation data associated with the one or more clinical trial sites; and generating predicted quantitative values informative of the performance of the one or more clinical trial sites by applying a trained federated learning model to the plurality of clinical operation data. The trained federated learning model is trained using a federated network. The federated network renders inaccessible a first dataset of a first party to the second party, and further renders inaccessible a second dataset of the second party to the first party.

### CROSS-REFERENCE TO RELATED APPLICATIONS

This application claims the benefit of U.S. Provisional Application No. 63/242,756, filed on Sep. 10, 2021, which is incorporated by reference herein.

### BACKGROUND

Site selection is the process of selecting clinical study sites for clinical trials (i.e., healthcare facilities and principal investigators) for industry-sponsored clinical trials. To support site selection for a particular study, standard machine learning (ML) approaches require training on data from multiple sponsors that is centralized in one location (e.g., multi-sponsor consortiums, etc.). To improve algorithm performance, additional data from historical clinical trials are needed.

Today, many clinical trials are outsourced, and data is often handled by external vendors (or contractors). However, many of these data are siloed by external vendors due to proprietary reasons or existing privacy agreements and are restricted from direct data sharing, resulting in a main challenge in training a model on external data due to privacy and proprietary concerns. Therefore, there is a need for a privacy-preserving approach that allows for training models using external data without the need for data sharing.

### SUMMARY

Embodiments of the invention disclosed herein involve implementing federated learning (FL) models to predict performance of clinical trial sites for site selection of one or more prospective clinical trials.

Federated learning (FL) is an approach that enables ML models to be trained on decentralized or siloed data, without the need for inter-institutional data sharing

As described herein, by using a federated network in the presently disclosed embodiments, each party can train a ML model on its own data, and exchange ML model weights and pointers via secure network connections for training and updating ML model parameters, resulting in a federated model (also referred to herein as a federated learning (FL) model) trained on multiple datasets of multiple parties (or sites) without exposure of the underlying, private data. The resulting federated model exhibits improved performance due to having been trained on additional data and further avoids overtraining and overfitting to one or more specific datasets.

As described herein, in the presently disclosed embodiments, systems and methods are used to train ML models (e.g., FL models) for clinical trial site enrollment prediction in a cross-institutional, privacy-preserving manner (e.g., using training data from one or more parties) to identify likely high-enrolling sites based on study- and site-level features, historical enrollment, and/or other clinical operations data. FL models and their predictions are useful for predicting future performance of clinical trial sites for specific disease indications. In various embodiments, a list of the predicted top performing clinical trial sites can be provided to appropriate stakeholders for inclusion in a subsequent clinical trial. The performance of these FL models and the utility of the predictions are improved in comparison to a standard ML model that trains data from one party (or data partner).

Disclosed herein is a method for predicting performance of one or more clinical trial sites for a prospective clinical trial, comprising: obtaining input values of a plurality of clinical operation data associated with the one or more clinical trial sites; and generating predicted quantitative values informative of the performance of the one or more clinical trial sites by applying a trained federated learning model to the plurality of clinical operation data, wherein the trained federated learning model is trained using a federated network, and wherein the federated network renders inaccessible a first dataset of a first party to the second party, and further renders inaccessible a second dataset of the second party to the first party.

In various embodiments, the one or more clinical trial sites comprises one or more clinical facilities and/or investigators.

In various embodiments, the plurality of clinical operation data comprise at least one of historical clinical trial performance, site characteristic(s), and site location(s), wherein the plurality of clinical operation data are associated with the same disease indication as that planned for the prospective clinical trial at the one or more clinical trial sites.

In various embodiments, the input values of the plurality of clinical operation data comprise at least one of NCT Number, site location, number of subjects consented, number of subjects enrolled in a trial, number of subject that completed a trial. Site Open Date (Or first patient in date), Study-Country last patient in data, and derived data associated with the one or more clinical trial sites.

In various embodiments, the predicted quantitative values informative of performance of the one or more clinical trial sites comprise at least one of site enrollment, site default likelihood, and site enrollment rate.

In various embodiments, the trained federated learning model is trained using training data obtained from at least part of the one or more clinical trial sites.

In various embodiments, the trained federated learning model is trained using training data obtained from one or more additional clinical trial sites, wherein at least one of the one or more additional clinical trial sites differ from the one or more clinical trial sites.

Additionally disclosed herein is a method for developing a federated learning model for improving prediction informative of performance of one or more clinical trial sites for a prospective clinical trial, comprising: performing, by a first party, data standardization on a first dataset; setting up at least a portion of a federated network comprising computer spaces or secure user interfaces for the first party and a second party; and generating an improved federated learning model for predicting performance of one or more clinical sites, wherein the improved federated learning model is trained at least in part by the first party using the first dataset and is trained at least in part by the second party using a second dataset, evaluating, by the first party, the improved federated learning model, wherein the first dataset is accessible to the first party while inaccessible to the second party, and wherein the second dataset is accessible to the second party while inaccessible to the first party.

In various embodiments, the method further comprises locally preprocessing by the first party, the first dataset, wherein the first dataset is preprocessed by applying a compiled code to split the first dataset into training, validation, and a holdout test set for evaluating the improved federated learning model.

In various embodiments, a source code from the compiled code are accessible to the first party while inaccessible to the second party.

In various embodiments, generating the improved federated learning model for predicting performance of one or more clinical sites comprises: sending, by the first party, parameters locally trained on the first dataset through the federated network: receiving, by the first party, parameters trained on the second dataset within the federated network; and updating, by the first party, the parameters of the locally trained federated learning model with the received parameters.

In various embodiments, generating the improved federated learning model for predicting performance of one or more clinical sites comprises: for each training epoch of a plurality of training epochs: sending, by the first party, parameters locally trained on the at least a portion of the first dataset through the federated network; receiving, by the first party, parameters from the second party that has trained the locally trained federated learning model using at least a portion of the second dataset; and updating, by the first party, the parameters of the locally trained federated learning model with the received parameters.

In various embodiments, generating the improved federated learning model for predicting performance of one or more clinical sites comprises: for each training epoch: receiving parameters from the second party that has individually trained the federated learning model using the second dataset; and averaging the parameters of the locally trained model with the received parameters.

In various embodiments, the federated network renders accessible the parameters of the locally trained federated learning model and the pointers of the first dataset to the second party, and renders inaccessible the first dataset to the second party.

Additionally disclosed herein is a method for developing a federated learning model for improving prediction informative of performance of one or more clinical trial sites for a prospective clinical trial, comprising: locally performing data standardization, by a second party, on a second dataset; setting up at least a portion of a federated network comprising secure compute spaces for each of the first party and a second party: receiving, by the second party, parameters of the federated learning model from the first party that has trained the federated learning model on a first dataset and pointers of a first dataset; locally training, by the second party, the received parameters of the federated learning model using a second dataset; sending, by the second party, the parameters trained on the second dataset to the first party, through the federated network, for further development of the federated learning model, wherein the first dataset is inaccessible to the second party, and wherein the second dataset is inaccessible to the first party.

In various embodiments, performing data standardization comprises receiving, by the second party, a compiled code from a first party; and aligning on input data and input features of the second dataset using the compiled code, wherein the compiled code masks proprietary engineered features and a source code.

In various embodiments, the federated network renders accessible the parameters of the locally trained federated learning model to the first party, and renders inaccessible the second dataset to the first party.

In various embodiments, the method further comprises locally preprocessing, by the second party, the second dataset, wherein the second dataset is preprocessed by applying the compiled code to split the second dataset into training, validation, and a holdout test set for evaluating the improved federated learning model.

In various embodiments, receiving parameters, locally training the received parameters, and sending the parameters is performed for each training epoch of a plurality of training epochs.

In various embodiments, locally training, by the second party, the received parameters of the federated learning model using a second dataset comprises locally training the received parameters over two or more training epochs.

In various embodiments, locally training the received parameters, and sending the parameters is performed for each training epoch of a plurality of training epochs.

In various embodiments, the federated learning model is developed using Python packages.

In various embodiments, the federated learning model comprises a machine learning model.

In various embodiments, the federated learning model comprises one of neural networks, XGboost, generalized linear models, regression models, classification models, random forest, and support vector machines.

In various embodiments, the federated training model comprises an improvement in identifying top-tier clinical trial sites.

In various embodiments, the first dataset comprises input values of a plurality of clinical operation data associated with the one or more clinical trial sites.

In various embodiments, the second dataset comprises input values of a plurality of clinical operation data associated with the one or more clinical trial sites.

In various embodiments, setting up at least a portion of the federated network comprises initiating a secure connection; and sending model parameters and data pointers through the secure connection, wherein the secure connection is initiated by sharing a connection string.

In various embodiments, the secure connection comprises a Pysyft duet.

In various embodiments, the secure connection is encrypted.

In various embodiments, the secure connection renders accessible the parameters of the locally trained federated learning model and the pointers of the first dataset to the second party, and renders inaccessible the first dataset to the second party.

In various embodiments, an architecture of the federated learning model is accessible to the first party while inaccessible to the second party.

In various embodiments, the first dataset comprises clinical operations data.

In various embodiments, the second dataset comprises clinical operations data.

In various embodiments, the federated learning model is further trained using a third dataset in a federated network, and wherein the federated network further renders inaccessible the third dataset to the first party and renders inaccessible the third dataset to the second party.

In various embodiments, the federated network comprises: a first walled computer space accessible to a first party, wherein the first walled computer space is inaccessible to the second party: a second walled computer space accessible to a second, wherein the second walled computer space is inaccessible to the first party; and a third walled computer space comprising a compiled code for processing the second dataset.

In various embodiments, each of the first and the second walled computer spaces comprises private access to data storage on a standalone virtual private cloud (VPC) environment or Amazon web service (AWS) S3 storage.

In various embodiments, the federated network relies on a central aggregation server for orchestrating model updates from each of the first party and the second party.

Additionally disclosed herein is a non-transitory computer readable medium for predicting performance of one or more clinical trial sites for a prospective clinical trial, comprising instructions that, when executed by a processor, cause the process to: obtain input values of a plurality of clinical operation data associated with the one or more clinical trial sites; and generate predicted quantitative values informative of the performance of the one or more clinical trial sites by applying a trained federated learning model to the plurality of clinical operation data, wherein the trained federated learning model is trained using a federated network, and wherein the federated network renders inaccessible a first dataset of a first party to the second party, and further renders inaccessible a second dataset of the second party to the first party.

In various embodiments, the one or more clinical trial sites comprises one or more clinical facilities and/or investigators.

In various embodiments, the plurality of clinical operation data comprise at least one of historical clinical trial performance, site characteristic(s), and site location(s), wherein the plurality of clinical operation data are associated with the same disease indication as that planned for the prospective clinical trial at the one or more clinical trial sites.

In various embodiments, the input values of the plurality of clinical operation data comprise at least one of NCT Number, site location, number of subjects consented, number of subjects enrolled in a trial, number of subject that completed a trial, Site Open Date (Or first patient in date). Study-Country last patient in data, and derived data associated with the one or more clinical trial sites.

In various embodiments, the predicted quantitative values informative of performance of the one or more clinical trial sites comprise at least one of site enrollment, site default likelihood, and site enrollment rate.

In various embodiments, the trained federated learning model is trained using training data obtained from at least part of the one or more clinical trial sites.

In various embodiments, the trained federated learning model is trained using training data obtained from one or more additional clinical trial sites, wherein at least one of the one or more additional clinical trial sites differ from the one or more clinical trial sites.

Additionally disclosed herein is a non-transitory computer readable medium for developing a federated learning model for improving prediction informative of performance of one or more clinical trial sites for a prospective clinical trial, comprising instructions that, when executed by a processor, cause the process to: perform, by a first party, data standardization on a first dataset: set up at least a portion of a federated network comprising computer spaces or secure user interfaces for the first party and a second party; and generate an improved federated learning model for predicting performance of one or more clinical sites, wherein the improved federated learning model is trained at least in part by the first party using the first dataset and is trained at least in part by the second party using a second dataset, evaluate, by the first party, the improved federated learning model, wherein the first dataset is accessible to the first party while inaccessible to the second party, and wherein the second dataset is accessible to the second party while inaccessible to the first party.

In various embodiments, the non-transitory computer readable medium further comprises instructions that, when executed by the processor, cause the processor to: locally preprocess by the first party, the first dataset, wherein the first dataset is preprocessed by applying a compiled code to split the first dataset into training, validation, and a holdout test set for evaluating the improved federated learning model.

In various embodiments, a source code from the compiled code are accessible to the first party while inaccessible to the second party.

In various embodiments, the instructions that cause the processor to generate the improved federated learning model for predicting performance of one or more clinical sites comprises instructions that, when executed by the processor, cause the processor to: send, by the first party, parameters locally trained on the first dataset through the federated network; receive, by the first party, parameters trained on the second dataset within the federated network; and update, by the first party, the parameters of the locally trained federated learning model with the received parameters.

In various embodiments, the instructions that cause the processor to generate the improved federated learning model for predicting performance of one or more clinical sites comprises instructions that, when executed by the processor, cause the processor to: for each training epoch of a plurality of training epochs: send, by the first party, parameters locally trained on the at least a portion of the first dataset through the federated network: receive, by the first party, parameters from the second party that has trained the locally trained federated learning model using at least a portion of the second dataset; and update, by the first party, the parameters of the locally trained federated learning model with the received parameters.

In various embodiments, the instructions that cause the processor to generate the improved federated learning model for predicting performance of one or more clinical sites comprises instructions that, when executed by the processor, cause the processor to: for each training epoch: receive parameters from the second party that has individually trained the federated learning model using the second dataset; and average the parameters of the locally trained model with the received parameters.

In various embodiments, the federated network renders accessible the parameters of the locally trained federated learning model and the pointers of the first dataset to the second party, and renders inaccessible the first dataset to the second party.

Additionally disclosed herein is a non-transitory computer readable medium for developing a federated learning model for improving prediction informative of performance of one or more clinical trial sites for a prospective clinical trial, comprising instructions that, when executed by a processor, cause the processor to: locally perform data standardization, by a second party, on a second dataset: set up at least a portion of a federated network comprising secure compute spaces for each of the first party and a second party; receive, by the second party, parameters of the federated learning model from the first party that has trained the federated learning model on a first dataset and pointers of a first dataset: locally train, by the second party, the received parameters of the federated learning model using a second dataset; and send, by the second party, the parameters trained on the second dataset to the first party, through the federated network, for further development of the federated learning model, wherein the first dataset is inaccessible to the second party, and wherein the second dataset is inaccessible to the first party.

In various embodiments, the instructions that cause the processor to perform data standardization comprises instructions that, when executed by the processor, cause the processor to: receive, by the second party, a compiled code from a first party; and align on input data and input features of the second dataset using the compiled code, wherein the compiled code masks proprietary engineered features and a source code.

In various embodiments, the federated network renders accessible the parameters of the locally trained federated learning model to the first party, and renders inaccessible the second dataset to the first party.

In various embodiments, the non-transitory computer readable medium further comprises instructions that, when executed by the processor, cause the processor to locally preprocess, by the second party, the second dataset, wherein the second dataset is preprocessed by applying the compiled code to split the second dataset into training, validation, and a holdout test set for evaluating the improved federated learning model.

In various embodiments, the instructions cause the processor to receive parameters, locally train the received parameters, and send the parameters is performed for each training epoch of a plurality of training epochs.

In various embodiments, the instructions cause the processor to locally train, by the second party, the received parameters of the federated learning model using a second dataset comprises instructions that, when executed by the processor, cause the process to locally train the received parameters over two or more training epochs.

In various embodiments, the instructions cause the processor to locally train the received parameters, and send the parameters is performed for each training epoch of a plurality of training epochs.

In various embodiments, the federated learning model is developed using Python packages.

In various embodiments, the federated learning model comprises a machine learning model.

In various embodiments, the federated learning model comprises one of neural networks, XGboost, generalized linear models, regression models, classification models, random forest, and support vector machines.

In various embodiments, the federated training model comprises an improvement in identifying top-tier clinical trial sites.

In various embodiments, the first dataset comprises input values of a plurality of clinical operation data associated with the one or more clinical trial sites.

In various embodiments, the second dataset comprises input values of a plurality of clinical operation data associated with the one or more clinical trial sites.

In various embodiments, the instructions that cause the processor to set up at least a portion of the federated network comprises instructions that, when executed by the processor, cause the processor to initiate a secure connection; and send model parameters and data pointers through the secure connection, wherein the secure connection is initiated by sharing a connection string.

In various embodiments, the secure connection comprises a Pysyft duet.

In various embodiments, the secure connection is encrypted.

In various embodiments, the secure connection renders accessible the parameters of the locally trained federated learning model and the pointers of the first dataset to the second party, and renders inaccessible the first dataset to the second party.

In various embodiments, an architecture of the federated learning model is accessible to the first party while inaccessible to the second party.

In various embodiments, the first dataset comprises clinical operations data.

In various embodiments, the second dataset comprises clinical operations data.

In various embodiments, the federated learning model is further trained using a third dataset in a federated network, and wherein the federated network further renders inaccessible the third dataset to the first party and renders inaccessible the third dataset to the second party.

In various embodiments, the federated network comprises: a first walled computer space accessible to a first party, wherein the first walled computer space is inaccessible to the second party; a second walled computer space accessible to a second, wherein the second walled computer space is inaccessible to the first party; and a third walled computer space comprising a compiled code for processing the second dataset.

In various embodiments, each of the first and the second walled computer spaces comprises private access to data storage on a standalone virtual private cloud (VPC) environment or Amazon web service (AWS) S3 storage.

In various embodiments, the federated network relies on a central aggregation server for orchestrating model updates from each of the first party and the second party.

Additionally disclosed herein is a system for predicting performance of one or more clinical trial sites for a prospective clinical trial, comprising: a computer system configured to obtain input values of a plurality of clinical operation data associated with the one or more clinical trial sites; wherein the computer system further generates predicted quantitative values informative of the performance of the one or more clinical trial sites by applying a trained federated learning model to the plurality of clinical operation data, wherein the trained federated learning model is trained using a federated network, and wherein the federated network renders inaccessible a first dataset of a first party to the second party, and further renders inaccessible a second dataset of the second party to the first party.

In various embodiments, the one or more clinical trial sites comprises one or more clinical facilities and/or investigators.

In various embodiments, the plurality of clinical operation data comprise at least one of historical clinical trial performance, site characteristic(s), and site location(s), wherein the plurality of clinical operation data are associated with the same disease indication as that planned for the prospective clinical trial at the one or more clinical trial sites.

In various embodiments, the input values of the plurality of clinical operation data comprise at least one of NCT Number, site location, number of subjects consented, number of subjects enrolled in a trial, number of subject that completed a trial. Site Open Date (Or first patient in date), Study-Country last patient in data, and derived data associated with the one or more clinical trial sites.

In various embodiments, the predicted quantitative values informative of performance of the one or more clinical trial sites comprise at least one of site enrollment, site default likelihood, and site enrollment rate.

In various embodiments, the trained federated learning model is trained using training data obtained from at least part of the one or more clinical trial sites.

In various embodiments, the trained federated learning model is trained using training data obtained from one or more additional clinical trial sites, wherein at least one of the one or more additional clinical trial sites differ from the one or more clinical trial sites.

Additionally disclosed herein is a system for developing a federated learning model for improving prediction informative of performance of one or more clinical trial sites for a prospective clinical trial, comprising: a first computer system configured to perform, by a first party, data standardization on a first dataset; and at least a portion of a federated network comprising computer spaces or secure user interfaces for the first party and a second party, wherein the first computer system generates an improved federated learning model for predicting performance of one or more clinical sites, wherein the improved federated learning model is trained at least in part by the first party using the first dataset and is trained at least in part by the second party using a second dataset, wherein the first computer system evaluates, by the first party, the improved federated learning model, wherein the first dataset is accessible to the first party while inaccessible to the second party, and wherein the second dataset is accessible to the second party while inaccessible to the first party.

In various embodiments, the computer system is configured to locally preprocess by the first party, the first dataset, wherein the first dataset is preprocessed by applying a compiled code to split the first dataset into training, validation, and a holdout test set for evaluating the improved federated learning model.

In various embodiments, a source code from the compiled code are accessible to the first party while inaccessible to the second party.

In various embodiments, generate the improved federated learning model for predicting performance of one or more clinical sites comprises: sending, by the first party, parameters locally trained on the first dataset through the federated network: receiving, by the first party, parameters trained on the second dataset within the federated network; and updating, by the first party, the parameters of the locally trained federated learning model with the received parameters.

In various embodiments, generate the improved federated learning model for predicting performance of one or more clinical sites comprises: for each training epoch of a plurality of training epochs: sending, by the first party, parameters locally trained on the at least a portion of the first dataset through the federated network; receiving, by the first party, parameters from the second party that has trained the locally trained federated learning model using at least a portion of the second dataset; and updating, by the first party, the parameters of the locally trained federated learning model with the received parameters.

In various embodiments, generate the improved federated learning model for predicting performance of one or more clinical sites comprises: for each training epoch: receiving parameters from the second party that has individually trained the federated learning model using the second dataset; and averaging the parameters of the locally trained model with the received parameters.

In various embodiments, the federated network renders accessible the parameters of the locally trained federated learning model and the pointers of the first dataset to the second party, and renders inaccessible the first dataset to the second party.

Additionally disclosed herein is a system for developing a federated learning model for improving prediction informative of performance of one or more clinical trial sites for a prospective clinical trial, comprising: a second computer system configured to locally perform data standardization, by a second party, on a second dataset; and at least a portion of a federated network comprising secure compute spaces for each of the first party and a second party, wherein the second computer system further receives, by the second party, parameters of the federated learning model from the first party that has trained the federated learning model on a first dataset and pointers of a first dataset, wherein the second computer system further locally trains, by the second party, the received parameters of the federated learning model using a second dataset, wherein the second computer system further sends, by the second party, the parameters trained on the second dataset to the first party, through the federated network, for further development of the federated learning model, wherein the first dataset is inaccessible to the second party, and wherein the second dataset is inaccessible to the first party.

In various embodiments, performing data standardization comprises receiving, by the second party, a compiled code from a first party; and aligning on input data and input features of the second dataset using the compiled code, wherein the compiled code masks proprietary engineered features and a source code.

In various embodiments, the federated network renders accessible the parameters of the locally trained federated learning model to the first party, and renders inaccessible the second dataset to the first party.

In various embodiments, the second computer system further locally preprocesses, by the second party, the second dataset, and wherein the second dataset is preprocessed by applying the compiled code to split the second dataset into training, validation, and a holdout test set for evaluating the improved federated learning model.

In various embodiments, the second computer system further receives parameters, locally train the received parameters, and send the parameters is performed for each training epoch of a plurality of training epochs.

In various embodiments, the second computer system further locally trains, by the second party, the received parameters of the federated learning model using a second dataset that comprises locally training the received parameters over two or more training epochs.

In various embodiments, the second computer system further locally trains the received parameters, and sends the parameters is performed for each training epoch of a plurality of training epochs.

In various embodiments, the federated learning model is developed using Python packages.

In various embodiments, the federated learning model comprises a machine learning model.

In various embodiments, the federated learning model comprises one of neural networks, XGboost, generalized linear models, regression models, classification models, random forest, and support vector machines.

In various embodiments, the federated training model comprises an improvement in identifying top-tier clinical trial sites.

In various embodiments, the first dataset comprises input values of a plurality of clinical operation data associated with the one or more clinical trial sites.

In various embodiments, the second dataset comprises input values of a plurality of clinical operation data associated with the one or more clinical trial sites.

In various embodiments, setting up at least a portion of the federated network comprises initiating a secure connection; and sending model parameters and data pointers through the secure connection, wherein the secure connection is initiated by sharing a connection string.

In various embodiments, the secure connection comprises a Pysyft duet.

In various embodiments, the secure connection is encrypted.

In various embodiments, the secure connection renders accessible the parameters of the locally trained federated learning model and the pointers of the first dataset to the second party, and renders inaccessible the first dataset to the second party.

In various embodiments, an architecture of the federated learning model is accessible to the first party while inaccessible to the second party.

In various embodiments, the first dataset comprises clinical operations data.

In various embodiments, the second dataset comprises clinical operations data.

In various embodiments, the federated learning model is further trained using a third dataset in a federated network, and wherein the federated network further renders inaccessible the third dataset to the first party and renders inaccessible the third dataset to the second party.

In various embodiments, the federated network comprises: a first walled computer space accessible to a first party, wherein the first walled computer space is inaccessible to the second party: a second walled computer space accessible to a second, wherein the second walled computer space is inaccessible to the first party; and a third walled computer space comprising a compiled code for processing the second dataset.

In various embodiments, each of the first and the second walled computer spaces comprises private access to data storage on a standalone virtual private cloud (VPC) environment or Amazon web service (AWS) S3 storage.

In various embodiments, the federated network relies on a central aggregation server for orchestrating model updates from each of the first party and the second party.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “model developing module 180A,” indicates that the text refers specifically to the element having that particular reference numeral. A reference numeral in the text without a following letter, such as “model developing module 180,” refers to any or all of the elements in the figures bearing that reference numeral (e.g., “model developing module 180” in the text refers to reference numerals “model developing module 180A” and/or “model developing module 180B” in the figures).

FIG. 1A depicts a system environment overview for predicting site performance for a prospective clinical trial, in accordance with an embodiment.

FIG. 1B depicts a block diagram of the site performance prediction system, in accordance with an embodiment.

FIG. 2A depicts a block diagram for predicting site performance for uses such as site selection in a prospective clinical trial, in accordance with an embodiment.

FIG. 2B depicts a flow diagram for predicting site performance for uses such as site selection in a prospective clinical trial, in accordance with an embodiment.

FIG. 3A is a flow process for performing federated training of a machine learning model across a first party and a second party, in accordance with an embodiment.

FIG. 3B is an interaction diagram for performing federated training of a machine learning model across a first party and a second party, in accordance with an embodiment.

FIG. 4 illustrates a first example federated network for use in federated training.

FIG. 5 illustrates a second example federated network for use in federated training.

FIG. 6A illustrates a first training paradigms used in federated training.

FIG. 6B illustrates a second training paradigms used in federated training.

FIG. 6C illustrates a third training paradigms used in federated training.

FIG. 7 illustrates an example computer for implementing the entities described herein.

FIG. 8 illustrates an example splitting of clinical operation data from multiple clinical trial sites for use in federated training.

FIG. 9 illustrates example results of site performance predictions trained on all-available data.

FIG. 10 illustrates example results of site performance predictions when non-enrolling sites were removed.

FIG. 11 illustrates example results of site performance predictions when FL models are trained with reduced local training data.

FIG. 12 illustrates example site ranking based on site performance predictions according to quartile-tiering of clinical trial sites.

FIG. 13 illustrates example improvement of site performances predicted from a FL model.

### DETAILED DESCRIPTION

### I. System Environment Overview

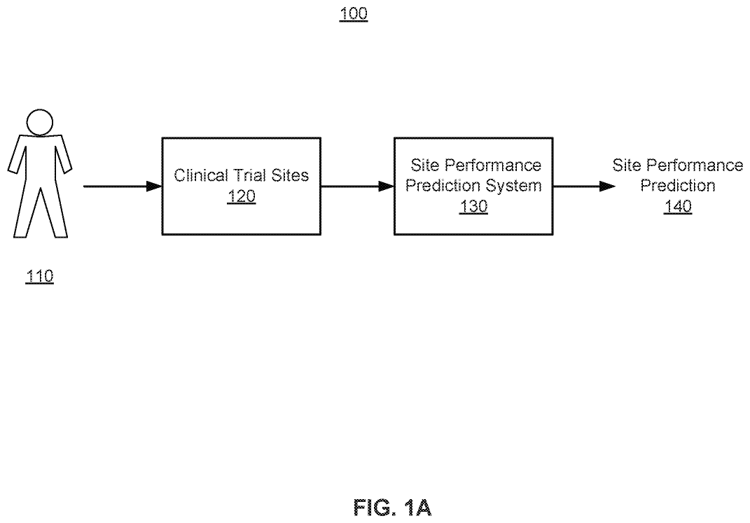

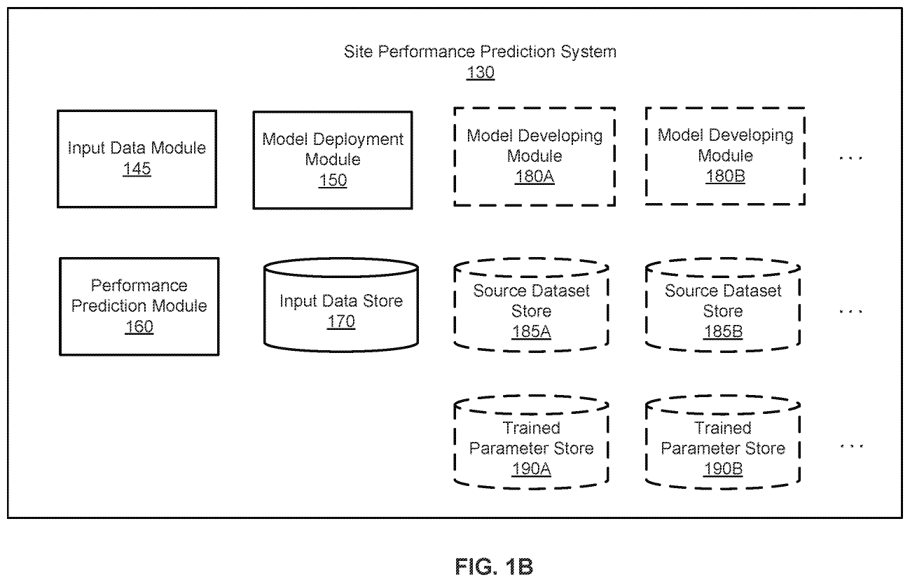

FIG. 1A depicts a system environment overview 100 for predicting site performance for a prospective clinical trial, in accordance with an embodiment. The system environment 100 provides context in order to introduce a subject (or patient) 110, clinical trial sites 120, and a site performance prediction system 130 for predicting a site's performance 140.

The system environment 100 may include one or more subjects 110 who were enrolled in clinical trials conducted by the clinical trial sites 120. In various embodiments, a subject (or patient) may comprise a human or non-human, human or non-human, whether in vivo, ex vivo, or in vitro, male or female, a cell, tissue, or organism. In various embodiments, the subject 110 may have met eligibility criteria for enrollment in the clinical trials. For example, the subject 110 may have been previously diagnosed with a disease indication. Thus, the subject 110 may have been enrolled in a clinical trial at a clinical trial site 120 that tested a therapeutic intervention for treating the disease indication. Although FIG. 1A depicts one subject 110, in various embodiments, the system environment overview 100 may include two or more subjects 110 that were enrolled in clinical trials conducted by the clinical trial sites 120.

The clinical trial sites 120 refer to locations that may have previously conducted a clinical trial (e.g., such that there are clinical operations data related to the previously conducted clinical trial). For example, the clinical trial sites 120 may have previously conducted one or more clinical trials that enrolled subjects 110. In various embodiments, one or more clinical trial sites 120 include at least one clinical facility and/or investigator that were previously used to conduct a clinical trial (e.g., in which the subjects 110 were enrolled) or can be used for one or more prospective clinical trials. In various embodiments, one or more clinical trial sites 120 are located in different geographical locations. In various embodiments, one or more clinical trial sites 120 generate or store clinical operations data describing the prior clinical trials (e.g., in which the subjects 110 were enrolled) that were conducted at the sites 120. In various embodiments, one or more clinical trial sites 120 were conducted for one or more different disease indications. Example disease indications include multiple myeloma, lung cancer, and/or other suitable disease indications in immunology, neuroscience, pulmonary hypertension, oncology, cardiovascular & metabolism, and infectious disease & vaccines.

The site performance prediction system 130 analyzes clinical operations data describing prior clinical trials from the one or more clinical trial sites 120 and generates a site performance prediction 140 by applying a federated model (e.g., a machine learning model trained via federated learning). The federated model involves a privacy-preserving approach (derived from federated learning) as described herein that allows for training machine learning (ML) models using both locally available datasets as well as external datasets without the need for data sharing (e.g., without need for sharing the locally available datasets or the external datasets). In various embodiments, the site performance prediction system 130 is a first party or is operated by a first party. In various embodiments, the site performance prediction system 130 applies a FL model to analyze or evaluate clinical operations data of the clinical trials sites 120, such as patient population availability, resources at the site, data collection procedures, site personnel-related qualities (e.g., interest and commitment, communicative skills, and experience) in conducting clinical trials, historical trial subject enrollment, historical trial subject screening, site operational data related to a clinical trial (e.g., site open date, first patient in, last patient in, etc.), and/or other key trial milestones for a clinical trial site, to generate a site performance prediction 140. In particular embodiments, the site performance prediction system 130 generates a site performance prediction 140 for a specific disease indication that is to be treated in a future clinical trial, the site performance prediction 140 identifying the likely best performing clinical trial sites for the specific disease indication.

In various embodiments, the site performance prediction system 130 includes or deploys a federated learning model that is trained using data from different parties (e.g., locally trained by a first party and externally trained by a second party such as external industry sponsors and/or contract research organizations (CROs), etc.). In some embodiments, the site performance prediction system 130 are trained by the same party (e.g., the first party). In some embodiments, the site performance prediction system 130 includes multiple datasets, wherein each dataset is locally available (or accessible) to one party.

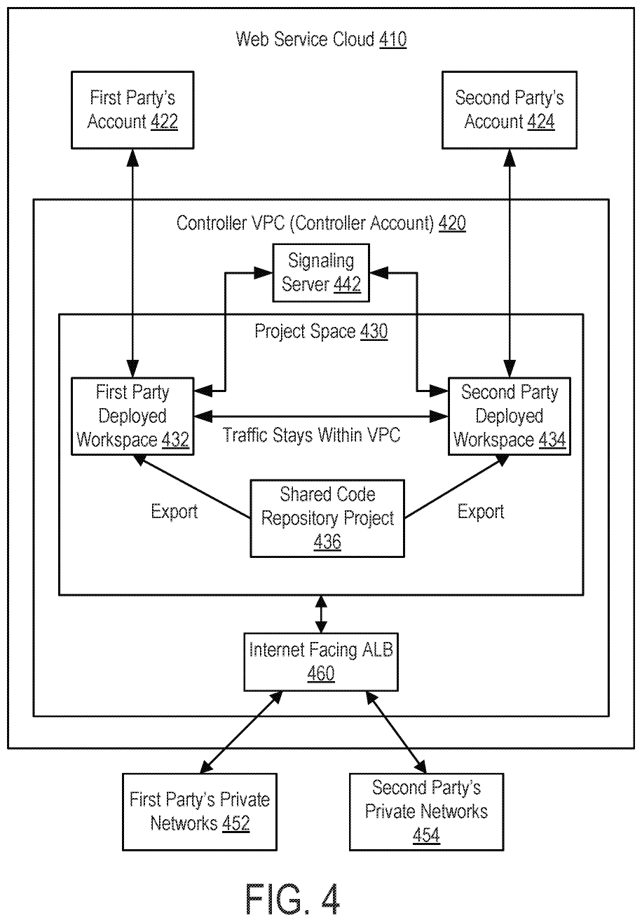

In various embodiments, the site performance prediction system 130 can include one or more computers, embodied as a computer system 400 as discussed below with respect to FIG. 4. Therefore, in various embodiments, the steps described in reference to the site performance prediction system 130 are performed in silico.

The site performance prediction 140 is generated by the site performance prediction system 130 and includes performance predictions of the clinical trial sites 120 for a prospective clinical trial. In various embodiments, the site performance prediction 140 is or includes a list of best performing sites for a prospective clinical trial. In various embodiments, the site performance prediction 140 is or includes at least 5 of the top-performing sites. In various embodiments, the site performance prediction 140 is or includes at least 10 of the top-performing sites. In various embodiments, the site performance prediction 140 is or includes at least 20 of the top-performing sites. In various embodiments, the site performance prediction 140 is or includes at least 50 of the top-performing sites. In various embodiments, the site performance prediction 140 is or includes a list of the worst performing sites for a prospective clinical trial, such that the site performance prediction 140 enables a recipient of the list to avoid enrolling the lowest performing sites for the prospective clinical trial. In various embodiments, the site performance prediction 140 is or includes at least 5 of the lowest-performing sites. In various embodiments, the site performance prediction 140 is or includes at least 10 of the lowest-performing sites. In various embodiments, the site performance prediction 140 is or includes at least 20 of the lowest-performing sites. In various embodiments, the site performance prediction 140 is or includes at least 50 of the lowest-performing sites. In various embodiments, the site performance prediction 140 can be transmitted to stakeholders so they can select sites for inclusion. In various embodiments, the site performance prediction 140 can be transmitted to principal investigators at the clinical trial site so they can determine whether to run the clinical trial at their site.

Reference is now made to FIG. 1B which depicts a block diagram illustrating the computer logic components of the site performance prediction system 130, in accordance with an embodiment. The components of the site performance prediction system 130 are hereafter described in reference to two phases. 1) a training phase and 2) a deployment phase. More specifically, the training phase refers to the building, developing, and training of federated models using training data across at least two parties. Therefore, the federated models are trained such that during the deployment phase, implementation of the federated models enables the generation of a site performance prediction (e.g., site performance prediction 140 in FIG. 1A).

As shown in FIG. 1B, the site performance prediction system 130 includes components for deployment including an input data module 145, a model deployment module 150, a performance prediction module 160, and an input data store 170.

Generally, the input data module 145 extracts input values of clinical operation data that may be obtained or stored in the input data store 170, and provides the input values to the model deployment module 150. The clinical operation data may include data from historical or new clinical trial sites for training or deploying a machine learning model to predict performance of the clinical trial sites for a prospective clinical trial. Obtaining the input values may encompass obtaining one or more clinical operation data from an external (e.g., publicly available) database or obtaining one or more clinical operation data from a locally available data store. Obtaining one or more clinical operation data can also encompass pulling the one or more clinical operation data from the external (e.g., publicly available) database or the locally available data store. In an embodiment, the inputs values may be obtained by receiving one or more clinical operation data, e.g., from a party that has performed the steps of obtaining the one or more clinical operation data from the external (e.g., publicly available) database or the locally available data store. Furthermore, the clinical operation data can be obtained by from memory of a storage memory. In another embodiment, the clinical operation data may be obtained from one or more locally available clinical operation data that are each pulled from a party at a single site. In such embodiments, the locally available clinical operation data is privately owned by the party at the single site.

The model deployment module 150 implements a trained FL model to analyze features of the extracted clinical operation data from clinical trial sites (e.g., clinical trial sites 120 in FIG. 1A) to predict performance of the clinical trial sites for a prospective clinical trial. The performance prediction module 160 generates predictions informative of the performance of the clinical trial sites.

Referring still to FIG. 1B, the site performance prediction system 130 may include modules implemented for training a federated model, such as model developing module 180A, source dataset store 185A, trained parameter store 190A, model developing module 180B, source dataset store 185B, trained parameter store 190B, and/or additional training components e.g., if there are additional parties or additional sites of a second party participating in the federated training of a federated model. Generally, there may be separate model developing modules (e.g., 180), separate source dataset stores (e.g., 185), and/or separate trained parameter stores (e.g., 190) for each separate party (or site) that is involved in training of a federated model. For example, if there are 2 parties that are involved in training a federated model, there may be 2 separate model developing modules, 2 separate source dataset stores, and/or 2 separate trained parameter stores. In another example, if there are 3 sites that are involved in training a federated model, there may be 3 separate model developing modules, 3 separate source dataset stores, and/or 3 separate trained parameter stores.

In various embodiments, the site performance prediction system 130 need not include the model developing module 180A, source dataset store 185A, trained parameter store 190A, model developing module 180B, source dataset store 185B, and trained parameter store 190B. For example, the model developing module 180A, source dataset store 185A, and trained parameter store 190A may be implemented by a different party and the model developing module 180B, source dataset store 185B, and trained parameter store 190B may be implemented by yet another different party. In various embodiments, the site performance prediction system 130 implements the model developing module 180A, source dataset store 185A, and trained parameter store 190A and a different party implements the model developing module 180B, source dataset store 185B, and trained parameter store 190B. In various embodiments, the site performance prediction system 130 initially includes the model developing module 180A, source dataset store 185A, trained parameter store 190A, model developing module 180B, source dataset store 185B, and trained parameter store 190B, and subsequently transmits the model developing module 180A, source dataset store 185A, and trained parameter store 190A to one party, and further transmits the model developing module 180B, source dataset store 185B, and trained parameter store 190B to another party. Thus, the two different parties can then train a FL model through federated learning methods.

In particular embodiments, the model developing module 180A may be used by a first party to train a FL model using a first dataset of clinical operation data that is stored in source dataset store 185A. The model developing module 180A outputs trained parameters of the FL model in the trained parameter store 190A. The trained parameters of the FL model in the trained parameter store 190A can be provided along with data pointers and then sent from the first party to the second party for further training the FL model. The pointers may comprise a variable that holds the address or location of another variable or function. For example, the data pointer may hold the address or location of data (e.g., training data as described herein), and can be used to provide the location of data to the model such that the model parameters can be updated during training of the model.

The FL model may be further trained by the model developing module 180B using a second dataset of clinical operation data in source dataset store 185B, resulting in the output of updated parameters in the trained parameter store 190B. Further description of the training of a federated model is described herein.

### II. Methods for Predicting Site Performance

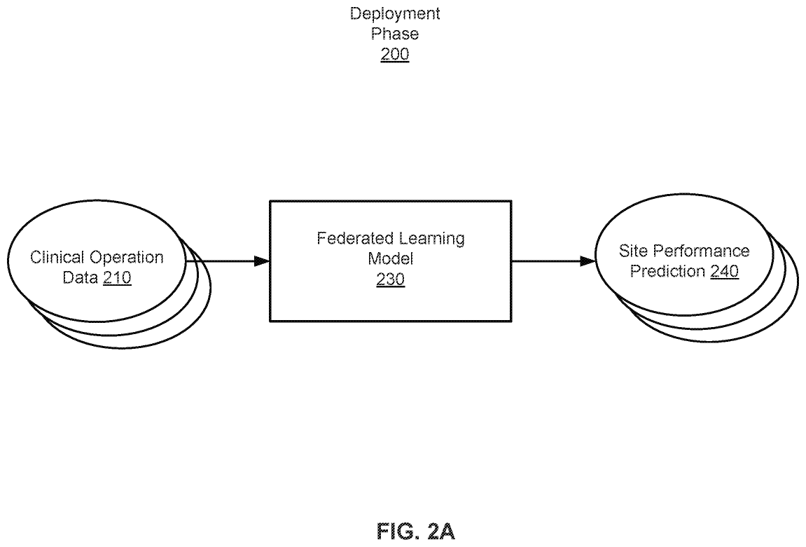

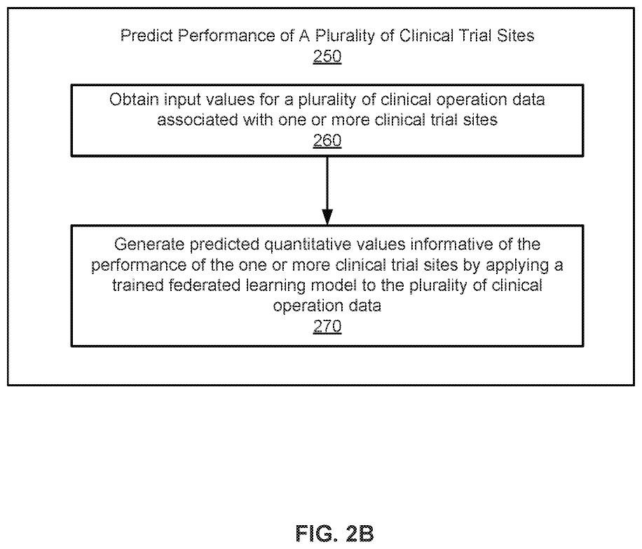

Embodiments described herein include methods for predicting performance of one or more clinical trial sites for a prospective clinical trial. Such methods can be performed by the site performance prediction system 130, such as by the input data module 145, the model deployment module 150, and the performance prediction module 160, as described in FIG. 1B. Reference will further be made to FIG. 2A, which depicts an example flow diagram 200 for predicting site performance, in accordance with an embodiment.

As shown in FIG. 2A, clinical operation data 210 may be obtained from one or more clinical trial sites (e.g., clinical trial sites 120 in FIG. 1A). In various embodiments, the clinical operation data 210 includes one or more datasets available to the same party (e.g., the first party). In various embodiments, the clinical operation data 210 includes multiple datasets available to different parties or different sites of a party and separately stored in different source dataset stores (e.g., source dataset stores 185A and 185B in FIG. 1B) For example, the clinical operation data 210 may include a first dataset stored in the source dataset store 185A and locally available (or accessible) to a first party and, a second dataset stored in the source dataset store 185B and available (or accessible) to a second party while inaccessible to the first party, and/or additional datasets (or sites of a party) participating in the federated training of the federated model 230.

In various embodiments, the clinical operation data 210 includes quantitative values of historical clinical trial performance (e.g., site enrollment), site characteristic(s), site location(s), protocol(s) for clinical trials, and/or disease indications. In various embodiments (e.g., a second party's dataset), a site enrollment starts from the date the first patient was screened or enrolled at a site (i.e., site first patient in date). In various embodiments (e.g., a first party's dataset), a site enrollment starts from the date the site was officially opened for enrollment. In various embodiments, the clinical operation data 210 include clinical trial data conducted for the same disease indication as that planned for the prospective clinical trial. In various embodiments, a site enrollment ends at the date the last patient was screened or enrolled (e.g., within a country).

The clinical operation data 210 (e.g., site enrollment data) can be processed using an input data module (e.g., input data module 145 in FIG. 1B) to generate input values for deploying the FL model 230. For example, the clinical operation data 210 may be initially obtained (e.g., in a data sheet obtained from an external or a local database) and stored in an input data store 170, and/or processed (e.g., extracted) by the input data module (e.g., input data module 145 in FIG. 1B) to generate input values of clinical operation data 210 for deploying the FL model 230. In particular embodiments, the input values of clinical operation data 210 may include raw data that is directly obtained from a data sources (e.g., first party, second party, site managers, other reporting structures for operational data, or publicly available database, etc.). For example, the input values of clinical operation data 210 may include site-level input values that are raw data directly obtained from a proprietary data source (e.g., first party, second party, etc.). As another example, the input values of clinical operation data 210 may include trial level input values (e.g., trial start date, trial end date, total enrolled, etc.) that are raw data directly obtained from a publicly available database (e.g., clinicaltrials.gov). In various embodiments, the input values of clinical operation data 210 includes at least one of NCT number, site location, number of subjects consented, number of subjects enrolled in a trial, number of subject that completed a trial, site open date (or first patient in date), study-country last patient in data, and/or derived data associated with the clinical trial sites for a prospective clinical trial. In another example, the input values of clinical operation data 210 includes at least 5 of NCT number, site location, number of subjects consented, number of subjects enrolled in a trial, number of subject that completed a trial, site open date (or first patient in date), study-country last patient in data, and/or derived data associated with the clinical trial sites for a prospective clinical trial. In another example, the input values of clinical operation data 210 includes at least 10 of NCT number, site location, number of subjects consented, number of subjects enrolled in a trial, number of subject that completed a trial, site open date (or first patient in date), study-country last patient in data, and/or derived data associated with the clinical trial sites for a prospective clinical trial.

The FL model 230 is applied to the input values of clinical operation data 210 using the model deployment module 150 (described in FIG. 1B) to generate site performance prediction 240. In various embodiments, the FL model 230 was previously trained on clinical operation data of one or more parties using a federated network, without a need to share the respective clinical operation data between the one or more parties. For example, the FL model 230 may be trained on clinical operation data stored by a first party, and then further trained on clinical operation data stored by a second party without a need to share the respective clinical operation data between the first party and the second party.

The site performance prediction 240 can be used to select sites for a prospective clinical trial. In various embodiments, the site performance prediction 240 includes quantitative values such as the predicted number of subjects a site is likely to enroll in a given study. Methods for determining a site performance prediction 240 are described herein.

Reference is now made to FIG. 2B, which depicts a flow diagram 250 for predicting site performance for uses such as site selection, in accordance with an embodiment.

At step 260, input values of a plurality of clinical operation data (e.g., clinical operation data 210 in FIG. 2A) associated with the one or more clinical trial sites are obtained (e.g., obtained using the input data module (e.g., input data module 145 in FIG. 1B)). In various embodiments, the input values of the plurality of clinical operation data are associated with the same disease indication as that planned for the prospective clinical trial. As described in FIG. 2A, the input values of the plurality of clinical operation data can, in various embodiments, include NCT number, site location, number of subjects consented, number of subjects enrolled in a trial, number of subject that completed a trial, site open date (or first patient in date), study-country last patient in data, and/or derived data associated with the one or more clinical trial sites.

At step 270, predicted quantitative values informative of the performance of the one or more clinical trial sites are generated by applying a trained FL model (e.g., FL model 230 in FIG. 2A) to the plurality of clinical operation data (e.g., clinical operation data 210 in FIG. 2A). The predicted quantitative values informative of performance of the one or more clinical trial sites can include one or more of site enrollment, site default likelihood, and/or site enrollment rate. In various embodiments, the predicted quantitative values informative of performance of the one or more clinical trial sites include site enrollment for the one or more clinical trial sites. In various embodiments, the predicted quantitative values informative of performance of the one or more clinical trial sites include site enrollment and site default likelihood for the one or more clinical trial sites.

In various embodiments, the one or more clinical trial sites can be evaluated and/or ranked according to the predicted site enrollment and predicted site default likelihood for each of the one or more clinical trial sites. In various embodiments, the one or more clinical trial sites are ranked according to the predicted site enrollment. For example, clinical trial sites that are predicted to enroll more patients in comparison to other clinical trial sites can be more highly ranked. In various embodiments, the one or more clinical trial sites are categorized into tiers. For example, the one or more clinical trial sites can be categorized into a first tier representing the best performing clinical trial sites, a second tier representing the next best performing clinical trial sites, and so on. In various embodiments, the one or more clinical trial sites are categorized into four tiers. In various embodiments, the top tier of clinical trial sites are selected and included in a prediction e.g., a site performance prediction 140 shown in FIG. 1A that can be provided to appropriate stakeholders for inclusion in a subsequent clinical trial.

### III. Training a Site Performance Prediction Model

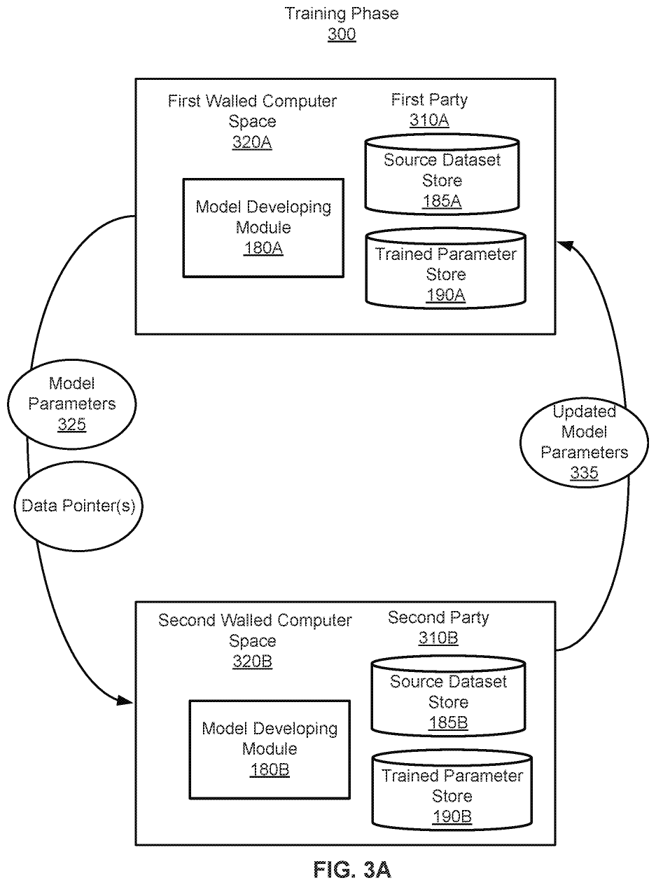

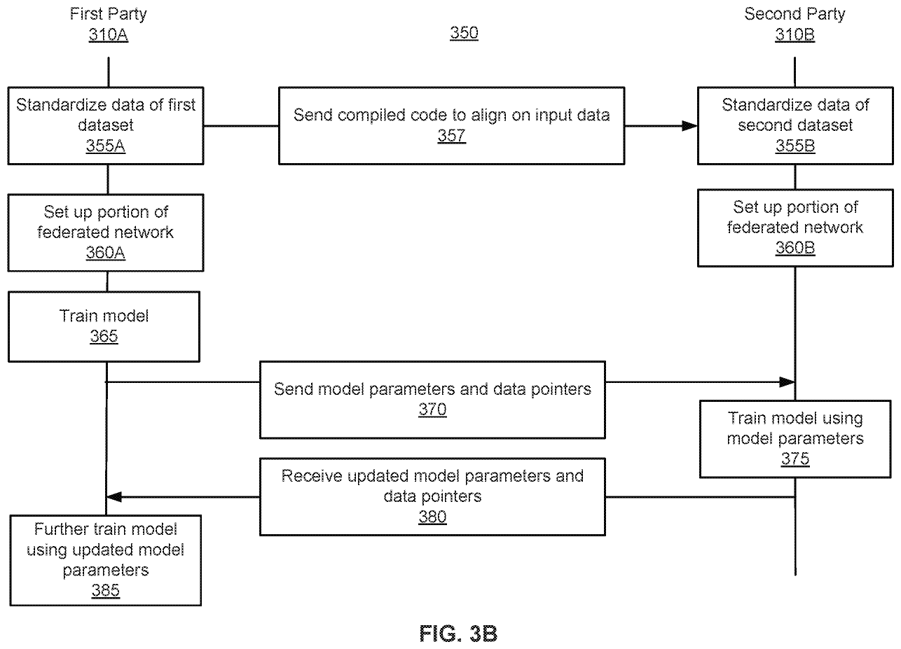

FIG. 3A depicts an example flow process for a training phase 300 in the site performance prediction system 130, in accordance with an embodiment. The training phase 300 includes a first party 310A, a second party 310B, and/or more additional parties. In various embodiments, the site performance prediction system 130 described above in reference to FIGS. 1A and 1B may also be operated by the first party 310A. As shown in FIG. 3A, each of the first party 310A and second party 310B include a model developing module 180, a source dataset store 185, and a trained parameter store 190. In various embodiments, there may be additional parties (e.g., third party, fourth party, etc.), and each of the additional parties include an individual model developing module, source dataset store, and trained parameter store (not shown). The first party 310A includes (or owns) the source dataset store 185A including a first dataset of clinical operations data, the model developing module 180A, and the trained parameter store 190A, as described in FIG. 1B.

In various embodiment, the source dataset store 185A is locally available to the first party 310A while inaccessible to other parties (or sites). In various embodiments, the source dataset store 185A includes a first dataset. In various embodiments, the first dataset may be obtained from a locally or publicly available database In various embodiments, the publicly available database can be clinicaltrials.gov. In various embodiments, the publicly available database can be DrugDev Data Query System (DQS). In various embodiments, a publicly available database is a library. In various embodiments, a publicly available database includes publicly available site enrollment and clinical operations data (e.g., from industry-sponsored clinical trials through multiple participating industry sponsors). In particular embodiments, the first dataset is a proprietary database from the first party.

In various embodiments, the source dataset store 185A includes a first dataset that includes a limited amount of data that can be used to train a machine learning model. Thus, implementing federated training by bringing in a second party enlarges the training dataset that is available to train the machine learning model (e.g., federated learning model). In various embodiments, the first dataset includes a limited amount of data pertaining to particular disease indications (e.g., multiple myeloma, lung cancer, and the like). In various embodiments, the first dataset includes data from less than 1000 clinical trial studies. In various embodiments, the first dataset includes data from less than 500 clinical trial studies In various embodiments, the first dataset includes data from less than 250 clinical trial studies. In various embodiments, the first dataset includes data from less than 100 clinical trial studies. In various embodiments, the first dataset includes data from less than 50 clinical trial studies. In various embodiments, the first dataset includes data from less than 25 clinical trial studies In various embodiments, the first dataset includes data from less than 20 clinical trial studies. In various embodiments, the first dataset includes data from less than 15 clinical trial studies. In various embodiments, the first dataset includes data from less than 10 clinical trial studies. In various embodiments, the first dataset includes data from less than 10,000 study-sites. In various embodiments, the first dataset includes data from less than 9000 study-sites. In various embodiments, the first dataset includes data from less than 8000 study-sites. In various embodiments, the first dataset includes data from less than 7000 study-sites. In various embodiments, the first dataset includes data from less than 6000 study-sites. In various embodiments, the first dataset includes data from less than 5000 study-sites. In various embodiments, the first dataset includes data from less than 4000 study-sites. In various embodiments, the first dataset includes data from less than 3000 study-sites. In various embodiments, the first dataset includes data from less than 2000 study-sites. In various embodiments, the first dataset includes data from less than 1000 study-sites. In various embodiments, the first dataset includes data from less than 750 study-sites. In various embodiments, the first dataset includes data from less than 500 study-sites. In various embodiments, the first dataset includes data from less than 250 study-sites. In various embodiments, the first dataset includes data from less than 100 study-sites. In various embodiments, the first dataset includes data from less than 50 study-sites. In various embodiments, the first dataset includes data from less than 25 study-sites. In various embodiments, the first dataset includes data from less than 10 study-sites.

The first party 310A may further include a FL model (e.g., FL model 230 in FIG. 2) as described herein. For example, during the training phase 300, the first party 310A may initialize the parameters of the FL model and begin the training of the FL model (e.g., by adjusting the initialized parameters of the FL model) using the first dataset within the first walled computer space 320A (such that the first dataset is not accessible by other parties e.g., the second party 310B). As another example, the first party 310A may include a FL model that is in the process of training. As another example, upon completion of the training phase 300, the first party 310A includes a fully trained FL model.

The second party 310B includes (or owns) the source dataset store 185B including a first dataset of clinical operations data (e.g., part of clinical operation data 210), the model developing module 180B, and the trained parameter store 190B, as described in FIG. 1B. In various embodiment, the source dataset store 185B is available to the second party 310B while inaccessible to other parties (or sites). For example, novel sites possessed by the second party may be permitted to have their identity revealed to the first party, but only predictions about that site from the global model are permitted to be used by the first party. In various embodiments, the source dataset store 185B include a second dataset. In various embodiments, the second dataset may be initially obtained from an external data owner. In various embodiments, a data owner is a hospital. In various embodiments, a data owner is a data storage company. In various embodiments, a data owner is an external industry sponsor. In various embodiments, a data owner is a contract research organization (CRO). In various embodiments, there may be one or more restrictions that prevent the second party 310B from sharing the second dataset to other parties (e.g., the first party 310A). In particular embodiments, the second dataset is a proprietary database from the second party.

In various embodiments, the first dataset and the second dataset include clinical operation data from US states for the modeling feature set due to inconsistencies between states/provinces reported internationally. In other embodiments, the first dataset and the second dataset includes clinical operation data from multiple countries.

In various embodiments, there are 3 components in the training phase 300: 1) data standardization, 2) federated network setup, and 3) federated model training and evaluation.

### III.A. Data Standardization

Prior to training, the first and the second parties may perform data standardization to align on the data to include for training, as well as common features. In various embodiments, the model developing module 180A standardizes data in the first party 310A, and the model developing module 180B standardizes data in the second party 310B prior to federated training. In various embodiments, the alignment process includes one or more of aligning on which trials to include in the training data set, deduplication of clinical trials that appear in both data sets, feature alignment, and target variable alignment. For example, feature alignment includes ensuring derived values were calculated in a similar manner in both data sets that are to be used to train the federated model. For example, the enrolling period of a site may be calculated differently in the two datasets because different starting dates were used. In such a case, the data standardization ensures that both parties agree on how the variable is calculated to enable federated training.

In various embodiments, feature alignment refers to aligning the features that are input into the federated model. For example, input values to a federated model can be structured as an input vector (e.g., [Input Value A, Input Value B, Input Value C, etc.]. Therefore, data standardization involves aligning of the features of the input vectors between the two parties, such that the federated training of the federated model across the two parties involves both parties providing similarly structured input vectors as input to train the federated model.

In various embodiments, prior to FL training and after data standardization, the data of the first party 310A and data of the second party 310B can be validated to ensure that the data standardization appropriately occurred. For example, the alignment of data elements between sources may be assessed using e.g., two trials that existed in each of the first and the second parties. The data elements that were compared include facility country & state/province, number of screened subjects, number of enrolled subjects, number of subjects who completed the trial, and/or site enrollment duration. The data definitions and/or values may be compared.

In various embodiments, the first party may provide a compiled code to the second party such that the second party can perform data standardization without accessing the underlying data that is standardized. For example, the first party may share a precompiled code (e.g., compiled into computer binary code unreadable by human programmers) with the second party to perform data standardization on the data of the second party. First, this ensures that the data of the second party are in the correct format for training the federated model. Second, this ensures that the underlying features that are selected to be provided as input for training the federal model are not revealed to the second party. In other words, the underlying features can serve as proprietary engineered features of the first party that are masked from the second party.

### III.B. Federated Network

In various embodiments, the FL model is trained using a federated network to create secure, private computing spaces for each party, respectively, which ensures the privacy-preservation of the data. The federated network is included in the site performance prediction system (e.g., site performance prediction system 130 in FIGS. 1A and 1B).

Generally, the federated network includes one or more walled computer spaces 320. The walled computer spaces 320 are server instances that are not connected to the public internet, such that they are private. An environment including one or more walled computer space 320, such as the federated network, can be built by a third-party user, and/or a data-contributing party, wherein each party has private access via single sign on to their respective walled instance. In particular embodiments, a walled space is a function provided by Amazon web service (AWS). A walled computer space 320 only allows access by one party, while preventing other parties from accessing the walled computer space 320. In various embodiments, the federated network includes a first walled computer space 320A accessible to a first party 310A while inaccessible to the second party 320B. The federated network further includes a second walled computer space 320B accessible to a second party 310B while inaccessible to the first party 310A. In various embodiments, each of the first and the second walled computer spaces includes private access to data storage on a cloud server. In various embodiments, the cloud server is a standalone virtual private cloud (VPC) environment or Amazon web service (AWS) S3 storage.

In various embodiments, the federated network further includes a third walled computer space including a compiled code provided by the first party for the second party to process the second dataset in source dataset store 2 185B. In various embodiments, the federated network relies on a central aggregation server for orchestrating model updates from each of the first party and the second party.

Generally, the first party 310A and the second party 310B can exchange information to enable federated training of a FL model without sharing the first dataset or the second dataset. As shown in FIG. 3A, the first party 310A may provide model parameters (e.g., parameters from the trained parameter store 190A) and/or data pointers to the second party 310B such that the second party 310B can train the federated model using data from the source dataset store 185B. Additionally, the second party 310B may provide updated model parameters (e.g., updated parameters from the trained parameter store 190B after training the federated model) to the first party 310A. In various embodiments, the data pointers include the address or location of the training data (e.g., the first dataset, the second dataset, etc.), but not the model parameters. Thus, the data pointers can be informative such that the FL model can access the appropriate training data for updating the model parameters of the FL model during training. In some embodiments, the model parameters and data pointers are exchanged between the first party 310A and the second party 310B via a secure connection between party server instances. The secure connection enables sharing of data pointers and model parameters during training, but not the underlying training data that is used to train the FL model. In various embodiments, the secure connection is a PySyft secure connection. PySyft is an opensource framework that creates encrypted connections between two parties (the Duet). The Duet is initiated by the sharing of a unique connection string between the two parties.

In various embodiments, the secure connections rely on a central aggregation server that orchestrates global model updates from each site that has trained the FL model. In various embodiments, a unique hash or site id can be used or created by a central orchestrator to find sites e.g., the intersection and novel sites within each federated dataset. In various embodiments, the secure connection is implemented using Pysyft (e.g., Pysyft Python packages) or other secure connections (e.g., Pytorch) with encryption in transit to create a communication tunnel. In various embodiments, the central aggregation server is separate from either the first party 310A and the second party 310B and can be operated by a different party altogether. In various embodiments, the central aggregation server is operated by the first party 310A. In various embodiments, the central aggregation server provides parameters to/from the first party 310A or to/from the second party 310B after one or more training epochs (e.g., training iterations). In particular embodiments, the central aggregation server provides parameters to or receives parameters from the first party 310A or provides parameters to or receives parameters from the second party 310B after each training epoch. In various embodiments, the central aggregation server provides parameters to or receives parameters from the first party 310A or provides parameters to or receives parameters from the second party 310B after X training epochs, where X is at least 1, at least 2, at least 3, at least 4, at least 5, at least 6, at least 7, at least 8, at least 9, at least 10 training epochs, at least 20 training epochs, at least 30 training epochs, at least 40 training epochs, at least 50 training epochs, at least 100 training epochs, at least 200 training epochs, at least 500 training epochs, at least 1000 training epochs, at least 2000 training epochs, at least 5000 training epochs, or at least 10,000 training epochs.

### III.C. Federated ML Model Training

Generally, a FL model in the presently disclosed embodiments can undergo federated training using data owned by two or more parties (e.g., pharmaceutical companies and/or other companies). Federated models are trained on decentralized or siloed data, without the need for inter-institutional data sharing. In a FL network of multiple data partners or organizations, ML model weights are sent via secure network connections to each data partner for training, where the weights/parameters are updated or averaged across partners, resulting in a federated model that has been trained on multiple data sets without exposure of the underlying, private data. For example, by including additional site-level enrollment data from third-party datasets (e.g., industry sponsors and/or CROs) using the FL model, ML models can be trained on the third-party's site enrollment data while maintaining privacy of the underlying data. The performance of the FL models may be compared with standard ML approach of using training data from one data partner.

The objective of the FL model is to predict site performance (target variable=number of enrolled patients per site) in an upcoming, planned clinical trial for a list of known sites. Training data used to train the federated model include historical clinical trial study and site data (e.g., site historical trial performance, site characteristics, country information) for studies in the same disease indication as the prospective (or upcoming) clinical trial.

Referring to FIG. 3A, the training process starts with the first party 310A which performs data standardization and/or data preprocessing (e.g., feature engineering and feature selection) of the first dataset that is stored in the source dataset store 185A. For example, preprocessing the dataset includes splitting the dataset into training, validation, and/or a holdout test set for evaluation of the FL model. In various embodiments, preprocessing and/or feature engineering can alternative be performed in a secure compute environment or docker image such that it can be shared on a compute instance without direct sharing of proprietary code with a second party.

The model developing module 180A of the first party 310A trains the FL model using the first dataset. Here, the model developing module 180A adjusts parameters of the FL model over one or more training epochs to generate output parameters of the FL model. The output parameters of the FL model can be stored in trained parameter store 190A. The first party 310A then sends the model parameters and/or a request to the second party to access the data pointers (or data locations) of the second dataset. The second party may accept this request, thus making the second dataset available for further training of the FL model.

The second party 310B trains parameters of the FL model over one or more training epochs using the second dataset stored in the source dataset store 185B. Specifically, the model developing module 180B adjusts the parameters over one or training epochs and stores the updated model parameters in trained parameter store 190B. In various embodiments, as is shown in FIG. 3A, the first party 310A sends the model parameters 325 to the second party 310B and the second party 310B updates the received model parameters 325. In other embodiments, the first party 310A need not send the model parameters 325 to the second party 310B. In such embodiments, the second party 310B may initialize model parameters for the FL model and adjusts the initialized model parameters. Thus, the second party 310B trains the parameters of the FL model independent of the training that was conducted by the first party 310A.

The second party 310AB sends the updated model parameters 335 to the first party 310A for improving the performance of the FL model.

In various embodiments, the first party 310A uses the updated model parameters 335 as the final parameters of the FL model. For example, referring again to FIG. 3A, the first party 310A may initialize parameters of the FL model and trains the FL model using the first dataset stored in source dataset store 185A. Here, the first party 310A trains the FL model over multiple training epochs using the available training data of the first dataset. Having trained the FL model using the available training data of the first dataset, the first party 310A sends the model parameters 325 to the second party 310B, which further updates the model parameters 325 by training the FL model using the second dataset of the source dataset store 185B. The second party 310B sends back the updated model parameters 335 to the first party 310A. Here, the updated model parameters 335 serve as the final parameters of the FL model, which has been trained on both the first dataset and the second dataset. This embodiment is further described below in the Examples and is referred to as the “Remote sequential” embodiment.

In another embodiment, the first party 310A trains the FL model (e.g., adjusts parameters of the FL model) over a single training epoch. The first party 310A sends the model parameters 325 to the second party 310B which trains the FL model (e.g., adjusts the received model parameters 325) over a single training epoch. The second party 310B sends the updated model parameters 335 following the single training epoch back to the first party 310A. The first party 310A can then further train the FL model by adjusting the updated model parameters 335 over the next training epoch. Thus, this embodiment represents a federated epoch-wise training process in which the first party 310A and the second party 310B exchange parameters on a per-epoch basis. This process of training and exchanging of parameters between the first party 310A and second party 310B is repeated until all training data of the first dataset in the source dataset store 185A and/or all training data of the second dataset in the source dataset store 185B is used. This embodiment is further described below in the Examples and is referred to as the “Epoch-wise” embodiment.

In various embodiments, the first party 310A combines model parameters 325 with the updated model parameters 335 from the second party 310B to generate final parameters of the FL model. For example, referring again to FIG. 3A, the first party 310A initializes parameters of the FL model and trains the FL model (e.g., adjusts parameters of the FL model) using the first dataset in the source dataset store 185A. Here, the first party 310A may train the FL model over a single training epoch or over multiple training epochs. The second party 310B initializes parameters of the FL and trains the FL model using the second dataset in the source dataset store 185B. Here, the second party 310B trains the FL model independent of the training that was conducted by the first party 310A. The second party 310B sends model parameters to the first party 310A such that the first party 310A combines the locally developed model parameters with the received model parameters from the second party 310B. In various embodiments, the first party 310A combines (e.g., averages) the locally developed model parameters with the received model parameters by determining average values of the locally developed model parameters and received model parameters. Thus, these average values can represent the final parameters of the FL model. This embodiment is further described below in the Examples and is referred to as the “Federated Averaging” embodiment.

In various embodiments, the first party 310A combines the locally developed model parameters with the received model parameters by determining a weighted combination of the locally developed model parameters and received model parameters. For example, the weighted combination can be based on the number of training epochs used to generate the model parameters. As a specific example, if more training epochs were used to generate the locally developed model parameters than the received model parameters, the locally developed model parameters can be more heavily weighted in determining the weighted combination in comparison to the received model parameters.

The first party 310A can further validate the performance of the final parameters of the FL model using a validation dataset (e.g., validation dataset that was split when the initial dataset was pre-processed). After training, the first party 310A may further evaluate the performance of the final parameters of the FL model using a hold-out test set (e.g., a hold-out test set of clinical trial study-site pairs).

FIG. 3B depicts an interaction diagram for developing a FL model across two parties for improving prediction informative of performance of one or more clinical trial sites for a prospective clinical trial, in accordance with an embodiment. In various embodiments, developing a FL model includes federated training using a federated network.

At step 355A, a first party performs standardization on a first dataset and aligns on input data and input features of the first dataset, as described above in FIG. 3A. The first dataset may be preprocessed to split the first dataset into training, validation, and a holdout test set for evaluating the improved FL model.

At step 357, the first party sends compiled code to the second party. In various embodiments, the source code of the compiled code are accessible to the first party while inaccessible to the second party. The compiled code enables the second party to perform data standardization.

At step 355B, a second party performs standardization on a second dataset, as described above in FIG. 3A. Specifically, the second party receives the compiled code from the first party, and aligns on input data and input features of the second dataset using the compiled code. In some embodiments, the compiled code masks proprietary engineered features and a source code from the second party.

At step 360A, at least a portion of a federated network including secure computer spaces and/or user interfaces for each of the first party and a second party is set up by e.g., the first party, and/or other suitable parties.

At step 360B, at least a portion of a federated network including computer spaces or secure user interfaces for each of the first party and a second party is set up by e.g., the second party, and/or other suitable parties.

Referring to steps 360A and 360B, setting up at least a portion of the federated network includes initiating a secure connection by sharing a connection string. The secure connection can be used to send or exchange model parameters and/or data pointers.

In various embodiments, the secure connection comprises a Pysyft duet. In various embodiments, the secure connection is encrypted. In various embodiments, the secure connection renders accessible the parameters of the locally trained FL model and the pointers of the first dataset to the second party, and renders inaccessible the first dataset to the second party.

At step 365, the FL model is trained at least in part by the first party using the first dataset. The training process is described in detail above in FIG. 3A.

At step 370, the first party sends parameters of the FL model locally trained on the first dataset, and data pointers of the first dataset to the second party through the secure connection by using the federated network.

At step 375, the second party receives the parameters of the FL model from the first party that has trained on the first dataset and pointers of the first dataset. The second party then locally trains the received parameters of the FL model using a second dataset.

At step 380, the second party sends updated parameters trained on the second dataset and pointers of the second dataset to the first party, through the federated network, for further development of the FL model.

In various embodiments, the second party 310 receives model parameters (e.g., step 370), locally trains the received parameters (e.g., step 375), and/or sends the parameters (e.g., step 380) for each training epoch of a plurality of training epochs. In other embodiments, the second party 310 receives model parameters (e.g., step 370) and locally trains the received parameters (e.g., step 375) over two or more training epochs prior to sending the parameters (e.g., step 380). In various embodiments, the second party 310 receives model parameters (e.g., step 370) and locally trains the received parameters (e.g., step 375) over three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more training epochs prior to sending the parameters (e.g., step 380). In particular embodiments, an optimal number of training epochs is a hyperparameter that is tuned during FL model training Thus, the optimal number of training epochs may vary (e.g., when training on different datasets). In particular embodiments, an optimal number of training epochs is between 10-15 epochs. In particular embodiments, a maximum number of training epochs is 20 epochs.

At step 385, the first party receives updated parameters trained on the second dataset within the federated network; and updates the parameters of the locally trained FL model with the updated parameters from the second party to generate a final FL model with improved prediction performance. The first party may also evaluate the improved FL model.

### IV. Example Federated Model

Generally, a federated model is structured such that it analyzes input values from clinical operation data, and predicts performance of clinical trial sites based on the input values. In various embodiments, the FL model is any one of a regression model (e.g., linear regression, logistic regression, or polynomial regression), decision tree, random forest, gradient boosted machine learning model, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, the FL model is one of a neural network, a XGboost model, a generalized linear model, a regression model, a classification model, a random forest model, extremely randomized trees, a support vector machine, or other suitable model. In particular embodiments, the FL model is a machine learning model. In particular embodiments, the FL model is a neural network model.

The FL model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In particular embodiments, the machine learning implemented method is a logistic regression algorithm. In particular embodiments, the machine learning implemented method is a random forest algorithm. In particular embodiments, the machine learning implemented method is a gradient boosting algorithm, such as XGboost. In various embodiments, the model is trained using supervised learning algorithms, unsupervised learning algorithms, semi-supervised learning algorithms (e.g., partial supervision), weak supervision, transfer, multi-task learning, or any combination thereof.

As described above in reference to FIG. 3A, parties (e.g., first party 310A and second party 310B) may exchange parameters of the FL model. Thus, this enables the first party 310A and second party 310B to train a FL model by adjusting the parameters of the FL model through federated learning. In various embodiments, the parameters of the FL model exchanged by the parties can include model parameters and/or hyperparameters. Examples of model parameters include weights associated with nodes in layers of neural network, support vectors in a support vector machine, node values in a decision tree, and coefficients in a regression model. In particular embodiments, the federated model is a neural network and therefore, model parameters are weights associated with nodes in layers of the neural network. Example hyperparameters include the learning rate, depth or leaves of a decision tree, number of hidden layers in a deep neural network, number of clusters in a k-means cluster, penalty in a regression model, and a regularization parameter associated with a cost function. In particular embodiments, the federated model is a neural network and therefore, hyperparameters include the number of hidden layers in a deep neural network. In various embodiments, the FL model is developed by using Python packages.

In various embodiments, the first party 310A or the second party 310B trains the FL model across one or more training epochs. For example, each training epoch can refer to the adjustment of the parameters of the FL model based on a single training example. The first party 310A or the second party 310B may then transmit the parameters of the FL model after the one or more training epochs. In various embodiments, the first party 310A or the second party 310B trains the FL model across two or more training epochs, three or more training epochs, four or more training epochs, five or more training epochs, six or more training epochs, seven or more training epochs, eight or more training epochs, nine or more training epochs, or ten or more training epochs after which the first party 310A or the second party 310B transmits the parameters of the FL model.

FIG. 4 depicts an example federated network for federated training (e.g., in training phase 300 in FIG. 3A). The federated network may be deployed in the context of a third-party web service cloud 410 (e.g., Amazon Web Services). The federated network as shown in FIG. 4 may be implemented in a secure environment to ensure the privacy-preservation of the data for each party (e.g., first party 310A or second party 310B). For example, the federated network may be implemented as a standalone web service environment with its own virtual private cloud (VPC) 420 operated by a third-party (i.e., not part of either the first party's virtual private cloud account 422 or the second party's VPC account 424 with the web service cloud 410). Each party (e.g., first party 310A or second party 310B) may have a walled private compute space (e.g., a deployed workspace 426, 428) within a project space 430 of the controller VPC 420, including a private access to data storage on each party's own virtual private cloud (VPC) environment 422, 424 or AWS S3 storage, which is inaccessible to the other parties. A first dataset may be stored in a first deployed workspace 432 of the project space 430 that was accessible to the first party but not the second party or additional parties, and a second data set was stored in a second party deployed workspace 434 of the project space 430 that may be accessible to the second party but not the first party or additional parties. An additional project 436 may be used as a shared repository for compiled code for e.g., data standardization or preprocessing.

The model parameters and data pointers (e.g., in FIG. 3A) may be exchanged via a signaling server 442 (e.g., a WebRTC server) and the shared code repository project 436, (wherein data traffic stays within a VPC 420) which provides a secure connection between party deployed workspaces 432, 434. In an embodiment, the exchange may occur via a framework that creates encrypted connections between two parties which allows for the sharing of data pointers (i.e., data locations) and ML model parameters during training, but not of the data itself. The framework may be initiated by the sharing of a unique connection string between the two parties. An example of a such a framework includes a PySyft Duet.

As shown in FIG. 4, different users on respective private networks 452, 454 (e.g., users of the first party 310A and/or users of the second party 310B) interface with their respective workspaces via the Internet facing application load balance (ALB) 460. Here, the private networks 452, 454 provide authentication for the users through assigned credentials that then enable access to the respective workspaces 432, 434.

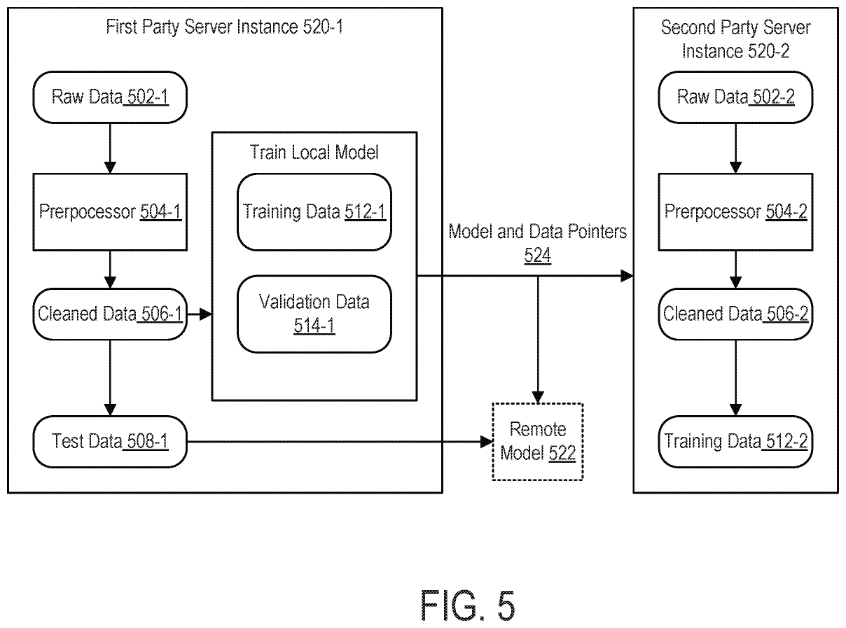

FIG. 5 illustrates a second example federated network for uses in federated training (e.g., training phase 300 in FIG. 3A). In some scenarios, the general modeling approach shown in FIG. 5 implements the federated network shown in FIG. 4. In other scenarios, the general modeling approach shown in FIG. 5 implements a different federated network.

The federated network, as shown in FIG. 5, included at least two server instances 520-1, 520-2, one for each party, hosted in e.g., an external cloud environment (e.g., AWS) that is not part of either party's virtual private cloud (VPC) accounts. Each party's dataset (including raw data 502, cleaned data 506, test data 508, training data 512, and a validation data 514) is stored in each party's respective cloud environment, and privacy-preserving hardware. The federated network configurations may be implemented to ensure no party could access the others' data storage or compute environment. The federated network may be implemented by instantiating a secure, encrypted connection between the server instances (e.g., using PySyft or similar framework described above), where only model weights (or model parameters) and data pointers were shared.

In an example scenario, the first party's dataset 502-1 may originate from a database containing site enrollment and clinical operations data from industry-sponsored clinical trials through a consortium of multiple participating industry sponsors. The second party's dataset 502-2 may originate from an industry platform that includes clinical and operational data (e.g., from over 23,000 clinical trials). For both parties' datasets 502-1, 502-2, clinical trial study data (i.e., start/end dates, inclusion/exclusion criteria) may be integrated from Clinicaltrials.gov. In an embodiment, only phase 2 and 3, and enrollment closed studies in adults are included in the dataset.

A task of the ML is to predict the number of subjects enrolled in a site in a given study using features derived from historical study site recruitment data, and/or additional study and site information for clinical trials of a disease (e.g., multiple myeloma). Each data sample may represent one study-site pair, such that studies with multiple sites included multiple rows, where sites that have participated in more than one study appear in more than one row.

Prior to FL training, alignment of data elements between sources may be assessed using e.g., two trials that existed in each party's data store. Data elements may be compared such as facility country & state/province, number of screened subjects, number of enrolled subjects, number of subjects who completed the trial, and site enrollment duration. Both the data definitions and their values may be compared.

Due to numerous inconsistencies between how states/provinces typically report internationally, an embodiment may consider only US states across data sources for the modeling feature set. In addition, the date the first patient was screened or enrolled at a site (i.e., site first patient in date) may mark the start of the site enrollment duration in e.g., the second party's data, whereas the date the site was officially opened for enrollment may be used in the first party's data. Both data sources may use the date the last patient was screened or enrolled within a country to mark the end of the site enrollment duration.

The guidelines may be configured such that substantial agreement (e.g., over 99%) agreement is observed for the majority of variables, and any differences are small relative to the mean values. After aligning data definitions and values, additional summary statistics may be compared between the full modeling data sets.

Next, federated learning models are trained. An example federated learning model may be a medium sized neural network model consisting of 4 layers with a ReLU activation layer used to fit the data. Model hyperparameter optimization may be performed on the local model only and the same hyperparameters may also be used for the FL model. All models may be trained for a fixed number of epochs to ensure model convergence and the model that produces the lowest validation mean-square error (MSE) may be evaluated on the test set 506-1.

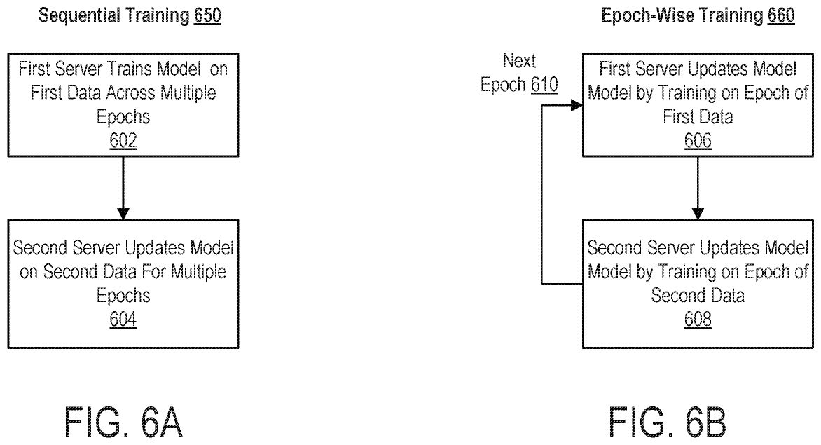

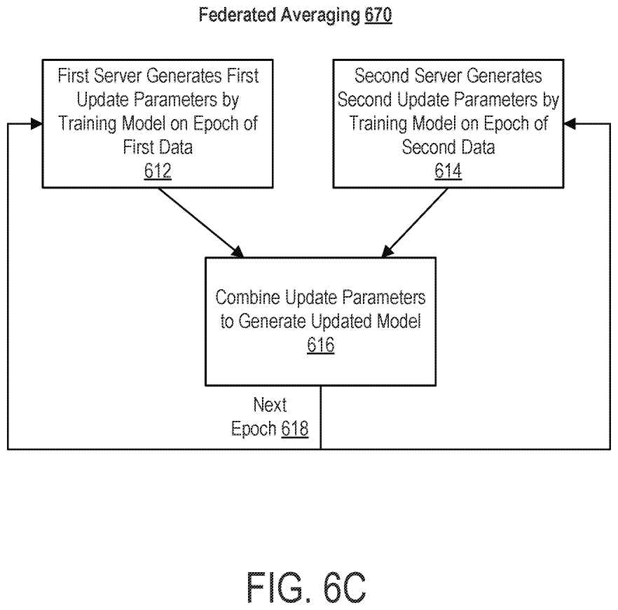

Following data preprocessing and data partitioning by the preprocessor 504-1, the general FL modeling approach as shown in FIG. 5 may include the following steps:
1) A local model is trained in the first party's (e.g., first party 310A in FIG. 3A) server instance 520-1 based on raw data 502-1 that is preprocessed via a preprocessor 504-1 to generate cleaned data 506-1, which is split into test data 508-1, training data 512-1, and validation data 514-1. In some scenarios, a best performing model is selected. In some scenarios, the model is selected by hyperparameter tuning.
2) Local model weights may be sent via a secure connection to the second party's 310B environment 520-2 for training on a second dataset (e.g., second dataset in the sourced dataset store 185B in FIGS. 1B and 3A). The second dataset is derived from raw data 502-2 that is pre-processed via a preprocessor 504-2 to generate cleaned data 506-2 used as training data 512-2. Only the model weights (or model parameters) are exchanged between the two servers 520 (e.g., via an exchange of model and data pointers 524). The model weights (or model parameters) may be updated in each training epoch, though the sequence of training data in which parameters are updated can vary. In an example implementation, three different training paradigms may be applied to test which was optimal for training the federated model, as shown in FIGS. 6A-C below.
3) A remote model 522 may furthermore be trained based on the test data 508-1 from the first part server instance 520-1. When remote model training is complete, the performance of the local and FL models may be evaluated on the test set.

Referring to FIG. 6A-C, three different training paradigms are illustrated including sequential training 650 (FIG. 6A), epoch-wise training 660 (FIG. 6B), and federated averaging (FedAvg) 670 (FIG. 6C). In sequential training 650, the local model is fully trained 602 on the first party's data (e.g., first dataset) by the first party server (across multiple epochs), and the local model parameters are then sent to the second party server to train on the remote data (e.g., second dataset) and to update 604 the local model parameters (which may then be shared with the first server for use by the first party). In epoch-wise training 660, random model weights are initialized, and for each epoch, the model is trained 606 on the first party's data and then trained 608 on second party's data to update the model parameters before proceeding to the next epoch 610. Therefore, the federated model parameters are updated after each epoch in epoch-wise training. In federated averaging (FedAvg) 670, random model weights were initialized, and for each epoch, the FL model is trained separately in each party or environment. For example, the first server generates 612 first update parameters based on an epoch of the first data and the second server generates 614 second update parameters by training on an epoch of the second data. The model weights of the two environments were then averaged or otherwise combined 616 at the end of each epoch. The process then proceeds 618 similarly with the next epoch. The three different federated model training paradigms (i.e., sequential training 650, epoch-wise training 660, and federated averaging (FedAvg)) 670 may be evaluated to determine a highest performing model. Additionally, different slices of data may be evaluated to understand e.g., scenarios where the FL model may outperform the locally trained model.

### V. Computer Implementation

The methods of the disclosed embodiments, including the methods of implementing federated models for predicting performance of clinical trial sites, are, in some embodiments, performed on one or more computers.

For example, the building and deployment of a federated learning model can be implemented in hardware or software, or a combination of both. In one embodiment of the invention, a machine-readable storage medium is provided, the medium comprising a data storage material encoded with machine readable data which, when using a machine programmed with instructions for using said data, is capable of executing the training or deployment of federated learning models and/or displaying any of the datasets or results described herein. The embodiments can be implemented in computer programs executing on programmable computers, comprising a processor, and a data storage system (including volatile and non-volatile memory and/or storage elements). Some computing components (e.g., those used to display the user interfaces may include additional components such as a graphics adapter, a display, a pointing device, a network adapter, at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information. The databases of the present invention can be recorded on computer readable media, e.g., any medium that can be read and accessed directly by a computer. Such media include, but are not limited to magnetic storage media, hard disc storage medium, and magnetic tape; optical storage media; electrical storage media such as RAM and ROM; and hybrids of these categories such as magnetic/optical storage media. Any convenient data storage structure can be chosen, based on the means used to access the stored information. A variety of data processor programs and formats can be used for storage, e.g., word processing text file, database format, etc.

In some embodiments, the methods of the invention, including the methods for predicting performance of clinical trial sites, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

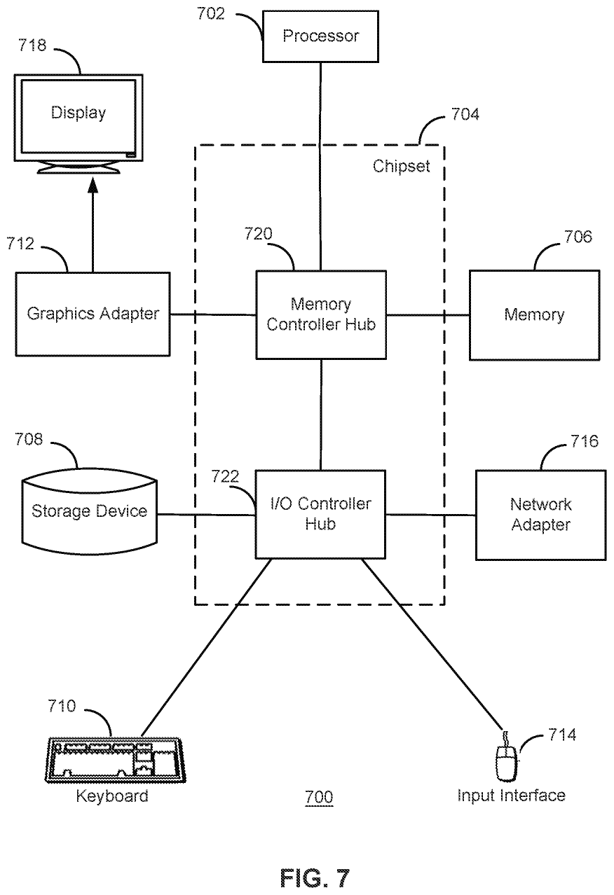

FIG. 7 illustrates an example computer for implementing various entities described herein. The computer 700 includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input interface 714, and network adapter 716 are coupled to the I/O controller hub 722. Other embodiments of the computer 700 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of pointing device, a keyboard, or some combination thereof, and is used to input data into the computer 700. In some embodiments, the computer 700 may be configured to receive input (e.g., commands) from the input interface 714 via gestures from the user. The network adapter 716 couples the computer 700 to one or more computer networks.

The graphics adapter 712 displays representation, graphs, tables, and other information on the display 718. In various embodiments, the display 718 is configured such that the user (e.g., data scientists, data owners, data partners) may input user selections on the display 718 to, for example, predict performance for a clinical trial site for a particular disease indication or order any additional exams or procedures. In one embodiment, the display 718 may include a touch interface. In various embodiments, the display 718 can show one or more predicted performances of a clinical trial site. Thus, a user who accesses the display 718 can inform the subject of the predicted performance of a clinical trial site.

The computer 700 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702.

The types of computers 700 used by the entities of FIG. 1A or 1B can vary depending upon the embodiment and the processing power required by the entity. For example, the site performance prediction system 130 can run in a single computer 700 or multiple computers 700 communicating with each other through a network such as in a server farm. The computers 700 can lack some of the components described above, such as graphics adapters 712, and displays 718.

### VI. Systems

Further disclosed herein are systems for implementing FL models for predicting performance of clinical trial sites. In various embodiments, such a system can include at least the site performance prediction system 130 described above in FIG. 1A. In various embodiments, the site performance prediction system 130 is embodied as a computer system, such as a computer system with example computer 700 described in FIG. 7.

### VII. Example 1: Federated Models Exhibit Improved Performance in Scenarios of Limited Training Data

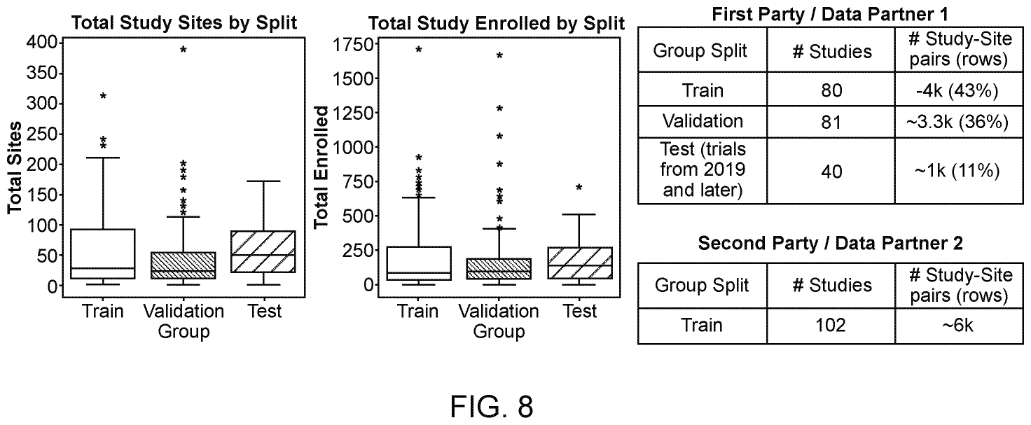

Reference is now made to FIG. 8, which depicts an example splitting of clinical operation data (e.g., clinical operation data 210 in FIG. 2A) from multiple clinical trial sites (e.g., clinical trial sites 120 in FIG. 1A) used to construct training, validation, or evaluation of a supervised machine learning (e.g., federated training as described in FIG. 3A, 4 or 5). As described in FIGS. 3A and 5, a dataset involved in the federated training (e.g., the first dataset of the first party 310A or the second dataset of the second party 310B) may be preprocessed, and then split into training, validation, and holdout test sets. Generally, the training set is used for training the FL model. The validation set is used during the training process to update the model parameters (e.g., after each training epoch). The holdout test set is used to evaluate a FL model that has been trained on the first dataset locally by the first party or a FL model that has been trained on the second dataset remotely by the second party.

As shown in FIG. 8, the first party's data which included 201 studies and ˜8,300 study-sites, was partitioned by study and split into training (43%), validation (36%), and test sets (11%), such that all sites from one study are included in the same split. The training and validation sets were randomly split 50-50 by study. The holdout test set include studies that were completed in 2019 or later to mimic the real-world use case generating predictions for prospective studies. All second party's data (102 additional studies, ˜6,000 study-sites) were used for FL training and were not split.

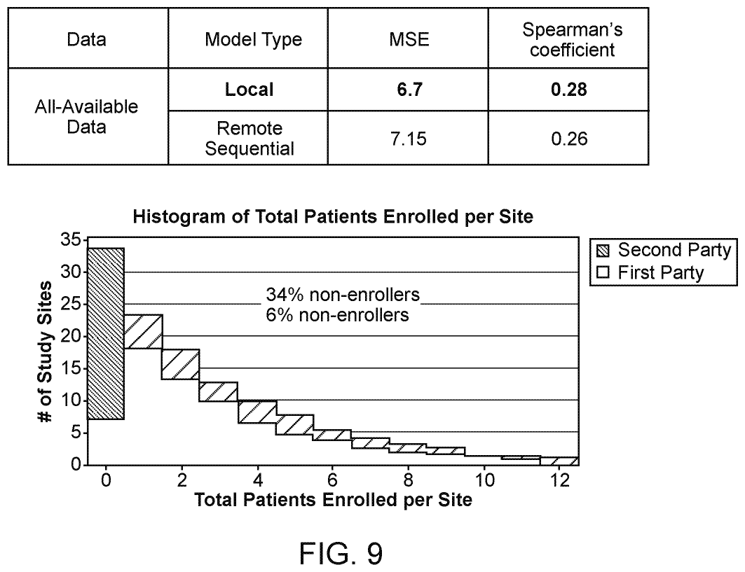

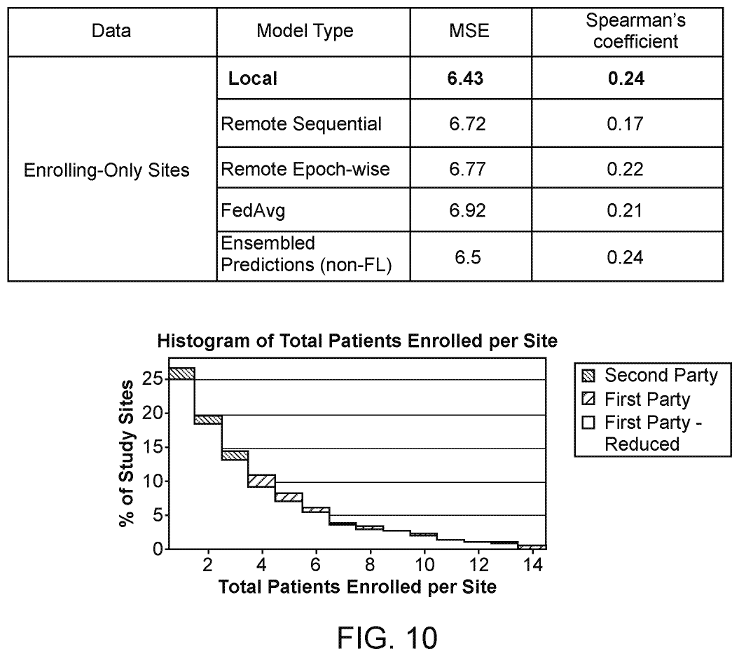

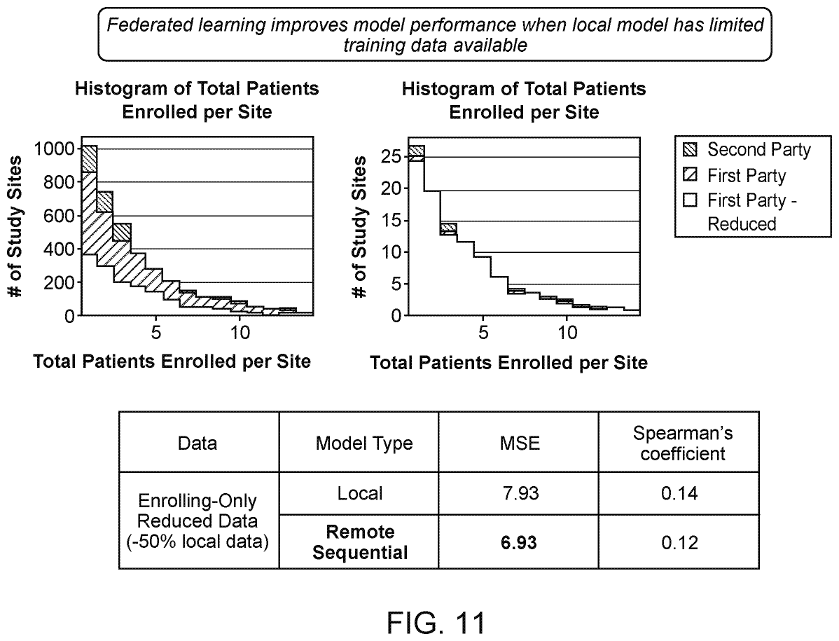

Reference now is made to FIGS. 9-11, in which the performance (e.g., % of study sites and total patients enrolled per site) of the local model and the FL model were compared by evaluating the holdout test set using mean squared error (MSE) and Spearman Rank correlation metrics, and depicted in histograms. In FIGS. 9-11, at least one of the three different training paradigms (e.g., sequential training, epoch-wise training, and federated averaging (FedAvg), as shown in FIG. 6A-C, were used during the federated training In each of FIGS. 9-11, the datasets for training included different slices of data. The different slices of data included any one of all-available data (FIG. 9), enrolling-only data (e.g., enrolling only sites in FIG. 10), or reduced enrolling-only data (FIG. 11). “All-available data” as shown in FIG. 9 refers to all local and remote data (e.g., first party's data and second party's data) used for training, including sites that enrolled zero patients (i.e., defaulted sites) and sites that enrolled one or more patients. “Enrolling-only data” as shown in FIG. 10 refers to a portion of the all-available data, where defaulted sites that have zero patients enrolled are removed from datasets of each party. “Reduced enrolling-only data” or “Enrolling-only reduced data” as shown in FIG. 11 indicates that the number of studies or study-site pairs in the local training data set is reduced by 50%, which mimics a common scenario where one party (e.g., a first party) may not have sufficient studies available in the local training data set for a particular disease indication (e.g., the disease indication where few industry sponsors have run interventional clinical studies).

The local and FL model performances described in FIGS. 9-11 for each experiment and FL training paradigm are reported in Table 1 below.

TABLE 1
Performance of local and FL models using different slices of data

| Data | Model Type | MSE | Spearman's coefficient |
|---|---|---|---|
| All-Available | Local | 6.70 | 0.28 |
| Data | Remote Sequential | 7.15 | 0.26 |
| Enrolling-Only | Local | 6.43 | 0.24 |
| Data | Remote Sequential | 6.72 | 0.17 |
| | Remote Epoch-wise | 6.77 | 0.22 |
| | FedAvg | 6.92 | 0.21 |
| | Ensembled | 6.5 | 0.24 |
| | Predictions | | |
| | (non-federated) | | |
| Enrolling-Only | Local | 7.93 | 0.14 |
| Reduced Data | Remote Sequential | 6.93 | 0.12 |
| (~50% local | | | |
| data) | | | |

Referring first to FIG. 9 and Table 1, using all-available training data, the FL model using sequential training achieved the lowest test error (e.g., mean squared error (MSE)) among the three different training paradigms (e.g., sequential training, epoch-wise training, and federated averaging (FedAvg)). However, the lowest test error of the FL model was outperformed by the local model, likely due in part to the inclusion of significantly more non-enrolling sites or non-enrollers (i.e., sites that enrolled zero subjects in a study) in the remote training data. For example, in a particular scenario of the study-pairs, as shown in FIG. 9, 6% of the first party's data and 33% of the second party's data is from non-enrolling sites. If the non-enrolling sites are removed from the dataset (FIG. 10), the distribution of site enrollment between the first party's dataset and the second party's dataset may be similar (e.g., data shown in FIG. 8). Furthermore, different rates of non-enrolling sites between training data sets may contribute to different (e.g., decreased) FL model performance.

Referring to FIG. 10 and Table 1, using data from enrolling-only sites, the performance of the local and FL model are improved compared to using all-available data (FIG. 9). Of the FL training paradigms, the sequential paradigm again demonstrated the best performance. However, the local model still exhibited lower test error than the FL model. As a non-federated comparison, the predictions made by a local model and a FL model fully trained on the second party's data alone are ensembled (i.e., averaged). The ensembled predictions perform relatively better than the FL model but do not achieve the performance of the local model.

Referring to FIGS. 11 and Table 1, using reduced enrolling-only data (e.g., “enrolling-only reduced data” in FIG. 11), the performance of local and FL models declined compared to using all enrolling-only data, while the FL model (“Remote Sequential”) achieved better performance than the local model. This demonstrates that in situations where a first party only possesses a limited amount of training data (which is often the case for certain disease indications), the federated learning approach disclosed herein is valuable for improving the performance of the federated learning model.

### VIII. Example 2: Federated Models Successfully Distinguish High Performing Sites (e.g., Quartile 1 Site) in Comparison to Low Performing Sites (e.g., Quartile 3-4 Sites)

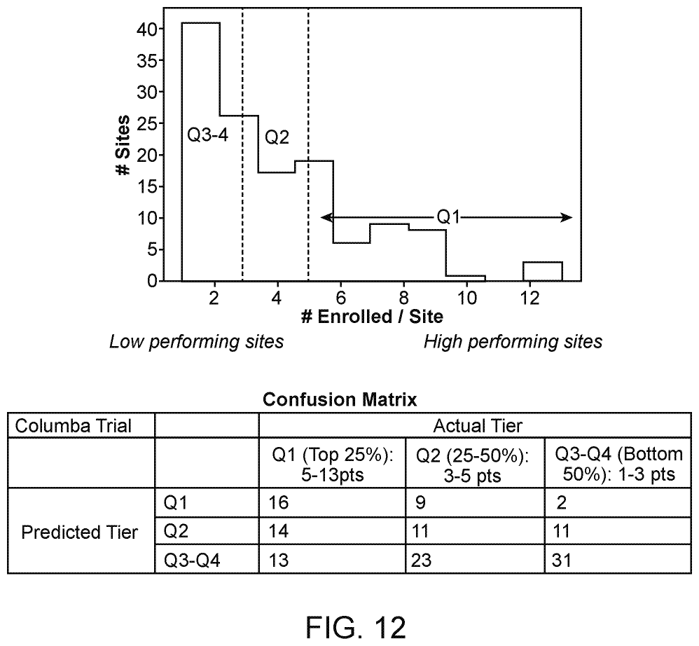

Reference is now made to FIG. 12, in which the effect of model predictions on site selection is assessed by classifying or ranking the predicted site performance (e.g., site enrollment) in each study in the holdout test set as performance-based tiers (e.g., site enrollment quartile) using a quartile-tiering approach. As shown in FIG. 12, a site enrollment quartile illustrates multiple sites classified into four tiers (e.g., Q1-Q4) that were ranked in an order based on the predicted number of patients enrolled in each site, in which number of sites in which the same predicted number of patients enrolled are grouped together. For example, Q1 (or the first quartile) represents a top tier including a portion of the studied sites that has best predicted performance (e.g., most numbers of patients enrolled in the site). Q4 represent a bottom tier including a portion of the studied sites that has lowest predicted performance (e.g., least numbers of patients enrolled in the site). Correct tier classification was assessed using the quartile tiers of site actual enrollment in the test studies. As shown by the Confusion Matrix in FIG. 12, the FL model was able to successfully categorize 16 Q1 clinical trial sites whose actual tier was Q1. Furthermore, accuracy, precision and/or recall were assessed for “top-tier” sites (e.g., the first quartile Q1 in FIG. 12) and/or “bottom-tier” sites (combined third and fourth quartiles Q3, Q4 in FIG. 12).

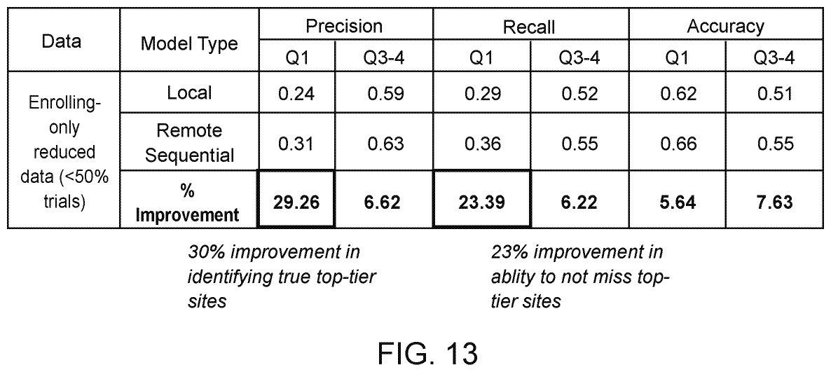

Referring to FIG. 13, for the reduced data scenario (e.g., FIG. 11), a real-world impact of model performance on site selection was evaluated. Generally, predictions generated by the FL model (“Remote Sequential”) are improved compared to predictions generated from the local model. Specifically, the FL model included an improvement of at least 20% precision or recall in identifying top-tier clinical trial sites (e.g., the first quartile Q1 in FIG. 12). In particular, for top-tier sites, the improvement in the performance predictions included ˜29% relative increase in precision, and ˜5.6% greater accuracy in identifying true top-performing sites. The FL model also has about 23% increase in recall, indicating less missed opportunities for selecting top-performing sites. Furthermore, the highest improvement are shown in recognizing top-tier sites (e.g., the first quartile Q1 in FIG. 12), while small increases in precision and recall were observed for bottom-tier (Q3-4).

These results demonstrate that FL is a viable approach for training ML models on siloed clinical operations data for the purpose of supporting clinical trial site selection, where this approach shows benefit over the local model when limited local training data is available. In practice, FL may show targeted improvement in predicting site enrollment for prospective clinical trials in disease areas with little local training data available through traditional consortiums. The gains in precision and recall of top-tier sites signify the potential to greatly reduce clinical trial costs and speed subject recruitment, ultimately benefiting patients in need of new therapies.

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1. A method for predicting performance of one or more clinical trial sites for a prospective clinical trial, comprising:
obtaining input values of a plurality of clinical operation data associated with the one or more clinical trial sites; and
generating predicted quantitative values informative of the performance of the one or more clinical trial sites by applying a trained federated learning model to the plurality of clinical operation data,
wherein the trained federated learning model is trained using a federated network, and
wherein the federated network renders inaccessible a first dataset of a first party to a second party, and further renders inaccessible a second dataset of the second party to the first party.

2. The method of claim 1, wherein the plurality of clinical operation data comprise at least one of historical clinical trial performance, site characteristic(s), and site location(s), wherein the plurality of clinical operation data are associated with the same disease indication as that planned for the prospective clinical trial at the one or more clinical trial sites.

3. The method of claim 1, wherein the input values of the plurality of clinical operation data comprise at least one of NCT Number, site location, number of subjects consented, number of subjects enrolled in a trial, number of subject that completed a trial, Site Open Date (Or first patient in date), Study-Country last patient in data, and derived data associated with the one or more clinical trial sites.

4. The method of claim 1, wherein the predicted quantitative values informative of performance of the one or more clinical trial sites comprise at least one of site enrollment, site default likelihood, and site enrollment rate.

5. A method for developing a federated learning model for improving prediction informative of performance of one or more clinical trial sites for a prospective clinical trial, comprising:
performing, by a first party, data standardization on a first dataset;
setting up at least a portion of a federated network comprising computer spaces or secure user interfaces for the first party and a second party;
generating an improved federated learning model for predicting performance of one or more clinical sites, wherein the improved federated learning model is trained at least in part by the first party using the first dataset and is trained at least in part by the second party using a second dataset; and
evaluating, by the first party, the improved federated learning model,
wherein the first dataset is accessible to the first party while inaccessible to the second party, and
wherein the second dataset is accessible to the second party while inaccessible to the first party.

6. The method of claim 5, further comprising locally preprocessing by the first party, the first dataset, wherein the first dataset is preprocessed by applying a compiled code to split the first dataset into training, validation, and a holdout test set for evaluating the improved federated learning model.

7. The method of claim 6, wherein a source code from the compiled code are accessible to the first party while inaccessible to the second party.

8. The method of claim 5, wherein generating the improved federated learning model for predicting performance of one or more clinical sites comprises:
sending, by the first party, parameters locally trained on the first dataset through the federated network;
receiving, by the first party, parameters trained on the second dataset within the federated network; and
updating, by the first party, the parameters of the locally trained federated learning model with the received parameters.

9. The method of claim 5, wherein generating the improved federated learning model for predicting performance of one or more clinical sites comprises:
for each training epoch of a plurality of training epochs:
sending, by the first party, parameters locally trained on the at least a portion of the first dataset through the federated network;
receiving, by the first party, parameters from the second party that has trained the locally trained federated learning model using at least a portion of the second dataset; and
updating, by the first party, the parameters of the locally trained federated learning model with the received parameters.

10. The method of claim 5, wherein generating the improved federated learning model for predicting performance of one or more clinical sites comprises:
for each training epoch:
receiving parameters from the second party that has individually trained the federated learning model using the second dataset; and
averaging the parameters of the locally trained model with the received parameters.

11. The method of claim 10, wherein the federated network renders accessible the parameters of the locally trained federated learning model and pointers of the first dataset to the second party, and renders inaccessible the first dataset to the second party.

12. A method for developing a federated learning model for improving prediction informative of performance of one or more clinical trial sites for a prospective clinical trial, comprising:
locally performing data standardization, by a second party, on a second dataset;
setting up at least a portion of a federated network comprising secure compute spaces for each of a first party and the second party;
receiving, by the second party, parameters of the federated learning model from the first party that has trained the federated learning model on a first dataset and pointers of a first dataset;
locally training, by the second party, the received parameters of the federated learning model using a second dataset; and
sending, by the second party, the parameters trained on the second dataset to the first party, through the federated network, for further development of the federated learning model,
wherein the first dataset is inaccessible to the second party, and
wherein the second dataset is inaccessible to the first party.

13. The method of claim 12, wherein performing data standardization comprises:
receiving, by the second party, a compiled code from a first party; and
aligning on input data and input features of the second dataset using the compiled code,
wherein the compiled code masks proprietary engineered features and a source code.

14. The method of claim 13, further comprising locally preprocessing, by the second party, the second dataset, wherein the second dataset is preprocessed by applying the compiled code to split the second dataset into training, validation, and a holdout test set for evaluating the improved federated learning model.

15. The method of claim 12, wherein the federated network renders accessible the parameters of the locally trained federated learning model to the first party, and renders inaccessible the second dataset to the first party.

16. The method of claim 12, wherein setting up at least a portion of the federated network comprises:
initiating a secure connection; and
sending model parameters and data pointers through the secure connection,
wherein the secure connection is initiated by sharing a connection string.

17. The method of claim 16, wherein the secure connection renders accessible the parameters of the locally trained federated learning model and the pointers of the first dataset to the second party, and renders inaccessible the first dataset to the second party.

18. The method of claim 12, wherein an architecture of the federated learning model is accessible to the first party while inaccessible to the second party.

19. The method of claim 12, wherein the federated learning model is further trained using a third dataset in a federated network, and
wherein the federated network further renders inaccessible the third dataset to the first party and renders inaccessible the third dataset to the second party.

20. The method of claim 12, wherein the federated network comprises:
a first walled computer space accessible to a first party, wherein the first walled computer space is inaccessible to the second party;
a second walled computer space accessible to a second, wherein the second walled computer space is inaccessible to the first party; and
a third walled computer space comprising a compiled code for processing the second dataset.
